(19) United States
(12) Patent Application Publication
(10) Pub. No.: US 2026/0363913 A1
(43) Pub. Date: Nov. 19, 2026

(54) TISSUE IMPLANTS, DEVICES, AND METHODS FOR INCREASING AQUEOUS DRAINAGE OF THE EYE

(71) Applicant: Iantrek, Inc., Harrison, NY (US)

(72) Inventors: Sean Buxton, Harrison, NY (US); Tsontcho Ianchulev, Harrison, NY (US); David Robson, Harrison, NY (US)

(21) Appl. No.: 19/680,629

(22) Filed: May 18, 2026

Related U.S. Application Data
(60) Provisional application No. 63/808,359, filed on May 19, 2025.
(60) Provisional application No. 63/930,429, filed on Dec. 3, 2025.

(51) Int. Cl. A61F 9/007; A61B 17/00; A61B 17/34

(52) U.S. Cl. CPC A61F 9/00781; A61B 2017/00367; A61B 2017/00969; A61B 17/3468; A61F 2210/0061

(57) ABSTRACT

A system for treating glaucoma including a tissue implant positionable within a dilated segment of Schlemm's Canal to facilitate aqueous outflow from an anterior chamber of an eye. The tissue implant is minimally-modified biological tissue that expands upon implantation within the dilated segment in any direction. Related methods, devices, tissue implants, and treatment systems are provided.

### CROSS-REFERENCE TO RELATED APPLICATIONS

This application claims the benefit of priority under 35 U.S.C. § 119(e) to U.S. Provisional Patent Application Serial Nos. 63/808,359, filed May 19, 2025, and 63/930,429, filed Dec. 3, 2025. The disclosures are incorporated by reference herein in their entireties.

### FIELD

The present disclosure relates generally to the field of ophthalmics, more particularly to tissue implants, devices, and methods for increasing aqueous drainage of the eye.

### BACKGROUND

Ophthalmic surgery is a treatment option for certain conditions of the eye, including glaucoma, which can result from elevated intraocular pressure. Surgical interventions can make use of an external approach (e.g., ab externo trabeculectomy, or shunting), which involves cutting the conjunctiva and the sclera to penetrate the eye to establish a trans-scleral outflow path, an ab interno trabecular approach (e.g., trans-scleral outflow stenting), which can utilize hardware-based implantable devices or non-implantable cutters to provide aqueous outflow from the eye, or an ab interno supraciliary approach, which can provide stenting using implantable non-biological hardware implants.

Current ab interno stenting devices and methods are primarily based on non-biological hardware materials such as polyimide, polyethersulphone, titanium, poly styrene-blocks-isobutylene-block-styrene and others. However, non-biological hardware-based implantable devices can lead to major erosion, fibrosis and ocular tissue damage such as endothelial cell loss.

New options for implantable devices useful in ophthalmic outflow strategies that present advantages over current implant technologies are needed, as are methods of procuring such devices efficiently and in a manner compatible with clinical workflows, e.g., for the treatment of glaucoma.

### SUMMARY

In an aspect, described is a system for treating glaucoma. The system includes a tissue implant positionable within a dilated segment of Schlemm's Canal to facilitate aqueous outflow from an anterior chamber of an eye. The tissue implant is minimally-modified biological tissue that, upon implantation within the dilated segment, expands more than 5% in any direction.

In an interrelated aspect, described is a device for dilating Schlemm's Canal including a distal portion sized for ab interno insertion into an anterior chamber of an eye. The distal portion includes an elongate, flexible shaft having a distal-most end sized to stretch Schlemm's Canal without tearing a trabecular meshwork as the distal-most end of the elongate, flexible shaft is advanced along Schlemm's Canal forming a dilated canal segment sized to receive a tissue implant, wherein the distal-most end is about 200-250 microns thick. The elongate, flexible shaft can be Nitinol or polyimide and has a cross-sectional shape that is rectangular or square. The elongate, flexible shaft optionally further includes a plurality of teeth proximal of the distal-most end formed on an outward surface of the distal portion. Each of the plurality of teeth has a leading surface and a trailing surface. The leading surface can be shaped to atraumatically slide along an outer wall of Schlemm's Canal during advancement and the trailing surface can be shaped to scrape the outer wall of Schlemm's Canal upon retraction of the distal portion from Schlemm's Canal.

In an interrelated aspect, described is a device for preparing Schlemm's Canal to receive a tissue implant that includes a distal portion sized for ab interno insertion into an anterior chamber of an eye. The distal portion has an elongate, flexible shaft including a tissue dilator proximal of a distal-most end of the shaft. During use, the elongate, flexible shaft is advanced along a circumferential contour of Schlemm's Canal. The tissue dilator has a dimension sized to stretch Schlemm's Canal without tearing a trabecular meshwork as the tissue dilator is advanced along Schlemm's Canal forming a dilated canal segment.

The trabecular meshwork tissue can be maintained substantially intact as the elongate, flexible shaft is advanced along the circumferential contour of Schlemm's Canal and/or as the shaft is withdrawn along the circumferential contour of Schlemm's Canal. The device can be part of a system that further includes the tissue implant. In use, the tissue implant can be positioned within at least a region of the dilated canal segment for aqueous outflow. The implant is minimally-modified biological tissue. The minimally-modified biological tissue can be scleral tissue, corneal tissue, or amniotic membrane tissue. The minimally-modified biological tissue can be dehydrated prior to implantation and upon implantation expands more than 5% in any direction upon hydration. The tissue implant in a dehydrated state can be about 50 microns-300 microns. The tissue implant in a rehydrated state can increase in size by about 5%-50%. The system can optionally include an implant delivery system having a delivery cannula, the tissue implant being contained within a lumen of the delivery cannula.

In an interrelated aspect, described is a method of increasing aqueous outflow through Schlemm's Canal including dilating at least a segment of Schlemm's Canal to stretch at least a portion of a trabecular meshwork. The method optionally includes inserting at least a first tissue implant within the segment of Schlemm's Canal, the first tissue implant being minimally-modified scleral allograft. The first tissue implant can expand upon deployment within the segment by more than 5% in any direction upon hydration to increase aqueous outflow from an anterior chamber through Schlemm's Canal. The method can optionally include inserting a second tissue implant within the segment of Schlemm's Canal, the second tissue implant being minimally-modified scleral allograft. Inserting the second tissue implant can involve positioning the second tissue implant a distance away from a first tissue implant previously positioned within the segment. The method can optionally include piercing at least a portion of the trabecular meshwork spanning between the first tissue implant and the second tissue implant. At least a distal end of the first tissue implant can be located within Schlemm's canal and a proximal end of the first tissue implant can be located within Schlemm's canal, within the trabecular meshwork, or within an anterior chamber. The proximal end of the first tissue implant can expand into the trabecular meshwork and/or into an anterior chamber upon rehydration and expansion following deployment. Dilating at least the segment of Schlemm's Canal can optionally include injecting a fluid into the segment. The fluid can be an ophthalmic viscoelastic solution.

In an interrelated aspect, described is a treatment system including one or more of a Schlemm's Canal dilation system and a tissue implant delivery system. The Schlemm's Canal dilation system can include a dilation tool having a proximal hand piece and an outer shaft coupled to and extending distal from the proximal hand piece. The outer shaft can be a tubular element having an inner lumen. The outer shaft can incorporate a straight proximal length extending along a longitudinal axis of the proximal hand piece and a curved distal length that curves away from the longitudinal axis. The curved distal length of the outer shaft can be about 15-20 mm long and the straight proximal length of the outer shaft can be about 50-55 mm long. The curved distal length can optionally incorporate a dual curve geometry. The outer shaft can include a distal tip forming a distal opening that has a heel, a distal point, and a length between the heel and the distal point. The distal tip can have a shape of a spatula sharpened to penetrate a trabecular meshwork. The distal tip can include a first region forming a first curve and a second region forming a second curve and an inflection between a convex shape of the first curve and a concave shape of the second curve. The Schlemm's Canal dilation system optionally further includes a fluid delivery device.

The tissue implant delivery system can include a delivery cannula; an inner member positioned internal to the delivery cannula; and a tissue implant held within the delivery cannula distal to the inner member. The tissue implant can be minimally-modified scleral allograft tissue. A distal end region of the delivery cannula can be flexible. The distal end region of the delivery cannula can take on a curved shape of the inner member. The distal end region of the delivery cannula can include a beveled distal opening. A heel of the beveled distal opening can be located on a radially-inward side of the curved shape. A heel of the beveled distal opening can be located on a radially-outward side of the curved shape creating a backward bevel direction. The backward bevel direction aids in preserving trabecular meshwork upon insertion of the distal end region into Schlemm's Canal.

The tissue implant delivery system further includes at least one actuator. The at least one actuator can optionally include a two-part actuator for deployment of the tissue implant from the delivery cannula. The at least one actuator can be configured to be pushed distally to retract the delivery cannula. The inner member can be fixed and the delivery cannula movable relative to the inner member.

In an interrelated aspect, described is a method of treating glaucoma including implanting a minimally-modified scleral allograft tissue implant that expands more than 5% in any direction upon hydration; and facilitating aqueous outflow from an anterior chamber of an eye with the tissue implant. The method can optionally include dilating Schlemm's Canal forming a dilated canal segment prior to the implanting. Dilating Schlemm's Canal can include advancing a dilation tool along at least a portion of Schlemm's Canal, the dilation tool with a distal end region having a dimension sized to stretch Schlemm's Canal without tearing a trabecular meshwork as the dilation tool is advanced along Schlemm's Canal. Dilating Schlemm's Canal can include positioning the minimally-modified scleral allograft tissue implant within Schlemm's Canal in a dehydrated state and forming the dilated canal segment as the implant hydrates.

In an interrelated aspect, described is a device for dilating Schlemm's Canal that includes a proximal hand piece having a slider; an outer shaft coupled to the proximal hand piece comprising a lumen having a distal opening; and an inner shaft positioned within the lumen of the outer shaft and movable relative to the outer shaft upon movement of the slider, wherein the inner shaft comprises a distal-most end. The distal-most end of the inner shaft is sized to dilate Schlemm's Canal as the distal-most end of the inner shaft is advanced through at least a portion of Schlemm's Canal forming a dilated canal segment. A trabecular meshwork of the dilated canal segment remains intact. The inner shaft has a cross-sectional shape taken transverse to a length of the inner shaft that can be square or rectangular. The inner shaft can include a first surface that is connected to a second surface by two lateral sides. In use, the first surface can be an inward-facing surface and the second surface is an outward-facing surface. A region of the first surface located proximal of the distal-most end of the inner shaft can optionally include one or more visual markers. In use, the one or more visual markers can indicate a degree of advancement of the inner shaft relative to Schlemm's Canal. The inner shaft optionally further includes a plurality of teeth located proximal of the distal-most end. The plurality of teeth can project from a region of the second surface. A thickness of the plurality of teeth along a lateral side from the first surface to the second surface can be about 140-150 microns. A thickness of the distal-most end of the inner shaft along a lateral side from the first surface to the second can be about 200-250 microns. The inner shaft further includes a neck region located proximal of the distal-most end. A thickness of the neck region along a lateral side from the first surface to the second surface can be less than the thickness of the distal-most end.

In some variations, one or more of the following can optionally be included in any feasible combination in the above methods, apparatus, devices, and systems. More details of the methods, apparatus, devices, and systems are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively but are intended to be illustrative. Also, relative placement of features and elements may be modified for the purpose of illustrative clarity.

FIG. 1 is a schematic view of a human eye showing a tissue implant being positioned in the eye in an example location of Schlemm's Canal;

FIG. 2A is a schematic view of a human eye showing two tissue implants being positioned in the eye in an example location of Schlemm's Canal;

FIG. 2B is a schematic view of a human eye showing two tissue implants of FIG. 2A and the trabecular meshwork cut;

FIGS. 2C-2D are schematic views showing positioning of an implant into Schlemm's Canal;

FIG. 3A is a box diagram illustrating a tissue system including a trephination system, a loading system, and an implant cartridge;

FIG. 3B is a box diagram illustrating a treatment system including a dilation system for Schlemm's Canal and an implant delivery system;

FIG. 4A is a perspective view of an implementation of an implant cartridge of a delivery device;

FIG. 4B is a perspective view of an implementation of a handpiece of a delivery device;

FIG. 4C is a perspective view of an implementation of a delivery device;

FIG. 4D is a perspective view of the implant cartridge of FIG. 4C taken along circle D-D with a tissue implant inside a delivery cannula;

FIG. 4E is the implant cartridge of FIG. 4D after the delivery cannula is retracted relative to a stopper revealing the tissue implant;

FIG. 4F is a perspective view of a distal end region of a delivery cannula having a beveled distal opening;

FIG. 4G is a side view of a distal end region of a delivery cannula having another implementation of a beveled distal opening;

FIG. 4H illustrates a cut line to create a backwards bevel as in FIG. 4G;

FIG. 4I is a perspective view of an implementation of an implant cartridge of a delivery device for use with any of the handpieces described herein;

FIG. 4J is a partially exploded view of an interior of the implant cartridge of FIG. 4I;

FIG. 4K is a partially exploded detail view of a carriage of a hand piece;

FIG. 4L is a view of the carriage of FIG. 4K having an implant cartridge loaded;

FIG. 4M is a perspective view of the carriage installed with the implant cartridge as in FIG. 4L and rotated at least partially around the longitudinal axis of the device;

FIG. 4N is a side view of the carriage installed with the implant cartridge as in FIG. 4M and further rotated around the longitudinal axis of the device;

FIG. 4O is a side view of the carriage installed with the implant cartridge as in FIG. 4N and further rotated around the longitudinal axis of the device;

FIG. 5A is a perspective view of an implementation of an implant cartridge of a delivery device;

FIG. 5B is a perspective view of an implementation of a handpiece of a delivery device;

FIG. 5C is a perspective view of an implementation of a delivery device incorporating the implant cartridge of FIG. 5A and the handpiece of FIG. 5B;

FIG. 5D is a detail view of the delivery device of FIG. 5C taken along circle D-D showing a scleral tissue implant;

FIG. 5E is a detailed view of the actuator of the delivery device of FIGS. 5B-5C that is slid proximally to retract the implant cartridge proximally;

FIG. 5F is another implementation of an actuator;

FIG. 5G is a cross-sectional view of an implementation of a handpiece of a delivery device as in FIG. 5B prior to depressing the actuator;

FIG. 5H is a cross-sectional view of the handpiece of FIG. 5G after depressing of the actuator;

FIG. 6A is a perspective view of another implementation of a delivery device having an actuator that is pushed distally to retract the implant cartridge proximally;

FIG. 6B is a perspective view of the delivery device of FIG. 6A with at least a portion of the housing removed to illustrate workings of the actuator;

FIG. 6C is a side view of the delivery device of FIG. 6B;

FIGS. 6D-6E are partial side views of other implementations of an actuator;

FIG. 7A is a perspective view of an implementation of a dilation tool in a first configuration;

FIG. 7B is a perspective view of the dilation tool of FIG. 7A in a second configuration;

FIG. 7C is a detail view taken at C-C of FIG. 7B of a distal end region of the dilation tool;

FIG. 7D is a detail view taken at C-C of FIG. 7B of a distal end region according to another implementation of the dilation tool of FIG. 7B;

FIG. 7E is a side view of an implementation of an inner shaft for the dilation tool of FIG. 7A;

FIG. 7F is a detail view of the outer shaft of FIG. 7E taken at circle B-B;

FIG. 7G is the detail view of the outer shaft of FIG. 7F taken at circle F-F and rotated 90 degrees;

FIG. 7H is a side view of an implementation of an outer shaft for the dilation tool of FIG. 7A;

FIG. 7I is a detail view of the outer shaft of FIG. 7H taken at circle B-B;

FIG. 7J is a side view of an implementation of an outer shaft of a dilation tool with an inner shaft extended outside the distal opening;

FIG. 7K is detail perspective view of the inner shaft of FIG. 7J.

It should be appreciated that the drawings are for example only and are not meant to be to scale. It is to be understood that devices described herein may include features not necessarily depicted in each figure.

### DETAILED DESCRIPTION

Disclosed are implants, devices, systems, and methods for increasing aqueous outflow from the anterior chamber of an eye and devices and systems for forming tissue implants for use in ophthalmic outflow procedures.

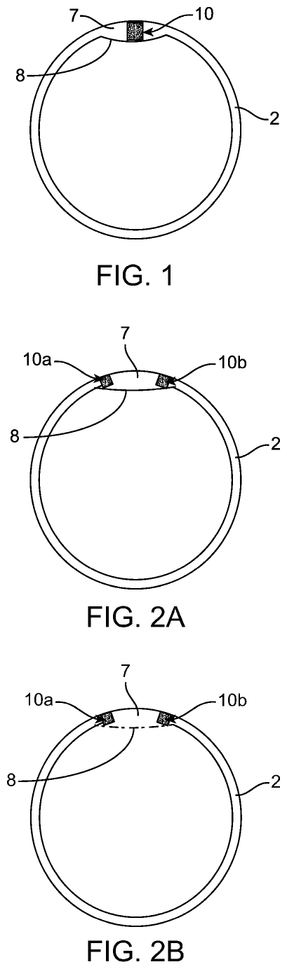

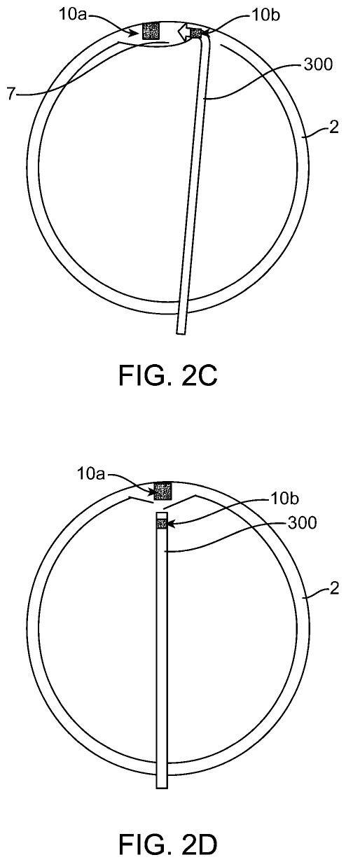

Freshly prepared bio-based (e.g., cell-based or tissue-based) stenting implants are advantageous in that they retain the original advantageous physical properties of the tissues used to produce them. However, the need to use such freshly prepared bio-based implants shortly after preparation can limit their utility, especially when the tissue used to fabricate the implants comes from an allogenic source and, even more critically, when the source material for implant fabrication cannot be obtained from living donor subject, as is the case with sclera-derived ophthalmic implants. In some implementations, a biologically-derived tissue material, such as a scleral tissue sample (e.g., a patch of scleral tissue material), is harvested or generated in vitro and formed into an implant, also referred to herein as a tissue implant, using a cutting device (e.g., a trephining device or trephination tool). In preferred implementations, the implant can be or can include an elongated body or strip of tissue or pellet, rod, bead, disk, etc. that does not have an internal lumen and is configured to maintain a space and provide scaffolding, stenting, and support of tissues, such as within a supraciliary or suprachoroidal space or stenting within another anatomical location such as within Schlemm's Canal or trans-scleral. As an example, FIG. 1 is a schematic view of Schlemm's Canal 2 of a human eye showing a tissue implant 10 positioned in an example location of Schlemm's Canal 2. FIG. 2A is a schematic view of Schlemm's Canal 2 of a human eye showing two tissue implants 10a, 10b positioned in an example location of Schlemm's Canal 2. The trabecular meshwork 8 is shown slightly expanded or stretched and spanning over the example location of the implants 10a, 10b. The dilated canal segment 7 formed by stretching Schlemm's Canal, such as with one or more of the dilation tools described herein, can include a trabecular meshwork 8 that is intact. Alternatively, the trabecular meshwork 8 of the dilated canal segment 7 can be disrupted after dilation with the dilation tool. FIG. 2B shows the trabecular meshwork 8 between the two tissue implants 10a, 10b in a dotted line indicating it has been removed, such as by cutting.

As disclosed herein, scleral tissue can be used for such tissue implants or another biological tissue, such as corneal tissue. Contrary to lumen-based devices (including many non-biological implants or stents formed of polymeric or metal materials), which can be limited by the lumen acting as a tract for fibrotic occlusion, scleral tissue implants described herein minimize fibrotic occlusion. Trephined scleral tissue can be preserved and stored for transport or later use and/or prepared for immediate use. Bio-based ophthalmic implants described herein can then be loaded into an implant delivery device and implanted into the eye via an ab interno delivery pathway to provide aqueous outflow from the anterior chamber. Accordingly, the implants described herein can be used as a phacoemulsification adjunct or as a stand-alone treatment to glaucoma as a micro-invasive glaucoma surgery (MIGS) treatment.

As will be described in more detail below, the implants described herein can be made from biologically-derived material that does not cause toxic or injurious effects once implanted in a patient, preferably the biologically-derived material is a tissue, such as scleral tissue or corneal tissue. The biological tissue preferably retains tissue-like properties after processing into an implant. For example, processed scleral tissue can retain its native microporosity allowing for superior host cellular infiltration and ingrowth upon deployment in an eye without the risks posed by synthetic materials having rigid synthetic edges, such as target site tissue erosion. Scleral tissue is naturally a relatively dense, fibrous connective tissue having collagen fibrils embedded in a matrix of proteoglycans and non-collagenous glycoproteins. Processing of harvested scleral tissue (e.g., dehydration) can leave microscopic air pockets or voids between the network of collagen fibrils, glycoproteins, and proteoglycans. The voids of the processed tissue readily reabsorb water upon deployment in the eye. Processing and cutting of the tissue does not alter the original relevant characteristic of the scleral tissue relating to the tissue's utility for reconstruction, repair, and/or replacement. Thus, the tissue implants described herein are minimally-modified and maintain the tissue-like properties of the natural source tissue. In contrast, a tissue that is ground up, formed into a paste for creating an implant and/or used to create an extract for use in an eye may still be considered a biomaterial, but the resulting implant is not a tissue.

Regardless of the source or type of tissue, the tissue can be cut (e.g., trephined) into a shape suitable for stenting and/or implantation in the eye. This cutting process of the tissue can be performed before the surgical implantation process or during the surgical implantation process. For example, the forming of the implant from the tissue can be performed at a tissue bank or manufacturing facility remote from the surgical suite where the implant would be implanted into a patient. In some embodiments, the implant can be pre-loaded within an implant cartridge (e.g., within a delivery cannula) and packaged for storage and transport at the manufacturing facility. The implant cartridge preloaded with the implant can be additionally designed to form part of the delivery system so that no transfer from the implant cartridge to another delivery tool is necessary.

It is advantageous to use an introducer portion of a delivery device (e.g., an elongate shaft portion of the delivery device, which may be a separate cartridge capable of being coupled to a delivery device handle) having a relatively small width, diameter, cross-sectional area, or gauge to introduce the prepared bio-based implant into a region of the eye. In some cases, using a thin introducer portion (e.g., elongate shaft) of a delivery device to install the implant into the eye can aid self-healing or closure of the access path into the eye (e.g., through a portion of the cornea) and may reduce discomfort for the subject. Therefore, it can be advantageous for an implant to be trephined or otherwise presented in a shape with a relatively narrow cross-sectional aspect (e.g., so that the implant will fit within a narrow cannula of an elongate portion of the delivery device). It is advantageous for the implant to retain its function for fluid outflow while also being shaped and sized to fit inside of a narrow introducer portion of a delivery device.

Ophthalmic implants prepared can have a smaller cross-sectional aspect (e.g., width, thickness, diameter, gauge, etc.) than a fresh tissue or, in some cases, than a rehydrated tissue implant, which can be advantageous for delivering the implant to the implant site. In some implementations, a tissue implant can increase in size with respect to one or more dimension (e.g., length, width, and/or thickness) after implantation at a target location of an eye, for example, as a result of in situ rehydration by a fluid of the eye. The tissue implant can be preserved for storage and the dimension(s) of the trephined implant selected to accommodate the physical size constraints of delivery into the eye while preserving the rehydrated physical, functional, and structural properties of the implant.

The methods of preparing an ophthalmic implant can include, constitute, or, in some cases, consist of a minimal structural modification of the biological tissue used to fabricate the implant. The minimal structural modification of the tissue can adjust a width of the tissue so as to form implants having a form factor that are pellets, rods and/or elongate strips suitable for insertion within an elongate delivery cannula. The elongate strips can have a length that is about 2-11 mm (e.g., a length greater than 3 mm) and a width that is less than 3 mm or less than 2 mm, for example, between 100-2000 microns. In addition to altering (e.g., decreasing) the length, width, and/or height (i.e., thickness) of the tissue using can also be modified by cutting a bio-based material described herein (e.g., an allogenic scleral tissue). In some implementations of a method, a cutting member (e.g., a plane) can be used to cut the tissue down to an appropriate thickness so as to better fit within an inner diameter of the delivery cannula. The tissue can be planed from 1,500 microns thick down to about 50 microns thick. Alternatively or additionally, a method described herein can be used to reduce or further reduce the length, the width, and/or the thickness of a trephined tissue implant to facilitate insertion of the implant within an inner diameter of a delivery cannula of a delivery device.

The minimal structural modification of the biological tissue (e.g., scleral tissue or corneal tissue or amniotic membrane) or other bio-tissue (cross-linked or not cross-linked) for implantable intraocular use can include a longitudinal trephination into an elongate strip of tissue having a width (or thickness) that is less than its length, for example, that can be more than 2 mm and less than 30 mm in length, as well as between about 0.1 mm and 2.0 mm in thickness, and between about 0.1 mm and 2.0 mm in width prior to loading within a delivery shaft. In some cases, a thickness of an elongate strip (e.g., of biologically-derived material following trephination and before processing) can be from 0.4 mm to 1.2 mm. In some implementations, the cutting of the bio-tissue can allow for adjustment of the width being cut and can simultaneously compress the bio-tissue to a consistent thickness.

The implant cut from the biologically-derived material (e.g., biological tissue) can be minimally manipulated, for example, by subjecting the trephined implant to compression, compaction, folding, rolling, exposure to one or more reagents, or another temporary manipulation that does not permanently affect the function of the processed biologically-derived material, allowing the material to return towards its original structure (e.g., when implanted at a target location of an eye). In some cases, the implant cut from the biologically-derived material (e.g., a biological tissue) can be minimally manipulated by subjecting a trephined tissue sample (e.g., a scleral tissue sample) to an ophthalmic implant preparation method described herein, which can reduce the size of the tissue sample. Thus, minimal manipulation of an implant fabricated from the biologically derived material (for instance, according to a perseveration method) can alter the size or shape of the cut tissue temporarily while still allowing the implant to resume the original relevant characteristics of the biologically-derived material, e.g., with respect to its utility for reconstruction, repair, or replacement after deployment. As an example, the biologically-derived material can be sclera that is cut into a shape that is oversized in relation to an inner diameter of a delivery tube through which the implant is implanted. The minimal manipulation of the cut implant can include subjecting the implant to one or more steps of a preparation method and/or temporarily compacting the scleral material into a lumen of the delivery shaft such that after implantation in the eye, the cut implant tends to return towards its original cut size.

Methods of fabrication, preparation, and/or implantation of implants formed from biologically-derived material are described herein in the context of being cut into an implant that can perform stent-like functions, such as maintaining a ciliary cleft or that can establish or maintain a fluid outflow pathway within the supraciliary space, the suprachoroidal space, and/or be implanted within a region of Schlemm's Canal (e.g., for increasing outflow of aqueous from anterior chamber of the eye). However, other methods and method steps are considered herein. For example, a method described herein can include compressing the biologically-derived material into a plug (e.g., to decrease one or more of a length, width, and/or thickness of the material of the implant) that is then implanted in a region of the eye for another purpose such as stenting, occlusion of traumatic ruptures, over-filtering bleb, posterior wall rupture, and other indications. In some cases, the implant preparation methods disclosed herein can achieve a change in size or shape of the trephined (e.g., cut) implant without mechanical manipulation. For example, a method of preparing the implant can include exposing the biologically-based material used to form the implant to one or more solutions, e.g., to decrease a size of the implant such that the implant can more easily be received within the delivery cannula, while still allowing the implant to expand to a larger size upon deployment and rehydration within the eye.

The implants (for example, formed from trephined biologically-derived material, such as scleral tissue or corneal tissue) can be loaded into a delivery channel for loading into a delivery device or portion thereof, such as an implant cartridge as described herein. In some implementations, it can be advantageous or, in some cases, necessary to decrease one or more dimensions (e.g., length, width, or thickness) of the implant in order to load the implant into a narrow gauge cannula, or other similarly shaped elongate delivery member that are useful or even necessary in applications described herein, which include delivery of the implant to a supraciliary space, ciliary cleft, or portion of Schlemm's canal of the eye. The cutting, preparatory treatment, loading, and transfer for delivery of the implant can be combined within a single assembly or can be performed by separate assemblies configured to work in conjunction with one another. One or more components of the assemblies described herein can be provided as a ready-to-use item. For example, the biologically-derived material used to form the implant can be pre-cut and prepared according to a method and provided within a preloaded shuttle assembly or implant cartridge that is sold as a ready-to-use component or a partially ready-to-use component that is coupled with a delivery hand piece, for example. The biologically-derived material used to form the implant can be cut and loaded at a site remote from the treatment facility, such as a tissue bank or manufacturing facility. In some implementations, a tissue implant prepared according to a method can be stored as a ready-to-use implant within a cartridge (e.g., introducer) that is configured to couple to a delivery tool at the treatment facility.

Preparation methods considered herein produce tissue implants that retain structural and functional features similar to those of (e.g., allogenic) freshly harvested biological tissue from which the implant is fabricated. This is especially advantageous because, by retaining such properties, the ophthalmic preparation methods described herein can qualify as “minimal manipulation” of the allogenic freshly harvested biological tissue, in view of regulatory oversight rules and definitions, which can substantially decrease the cost of fabrication and use of the implants. Moreover, the degree to which the size of the implant can be decreased (e.g., with respect to one or more of the implant's length, width, and/or thickness) using a method of preparation can allow or facilitate use of the prepared implant in smaller diameter (or gauge) cannulas, or elongate members of a delivery device (e.g., as compared to other implant technologies), which can be advantageous in applications of the implant involving ophthalmic implantation, which are frequently restricted with respect to the diameter or gauge of the delivery device used to deliver the implant in the eye.

A tissue sample for preparing the implant can be a biologically-derived tissue, such as a tissue obtained from a donor organism. In some cases, the biologically-derived tissue can be an allogenic tissue. In some cases, the biologically-derived tissue can be cadaver material, such as cadaver sclera or cornea. In various implementations, the tissue sample can be a scleral tissue sample (e.g., an allogenic scleral tissue sample). In some cases, the provided tissue sample can be provided on ice or otherwise cooled without freezing (e.g., storage in a refrigeration unit at or lower than about 4 °C, for instance between 0 °C and 4 °C). In some implementations, the provided tissue sample is provided preserved (e.g., dehydrated, frozen, lyophilized).

A method of preparing a tissue-based ophthalmic implant can include trephining (e.g., cutting) a provided tissue sample. In some implementations, a biological tissue sample can be trephined using a cutting device, for example, as described herein. In some implementations, a biological tissue sample can be trephined manually. In some cases, a biologically-derived material (e.g., a scleral tissue sample) can be trephined to a desired size and shape (e.g., an elongate shape having dimensions described herein) before commencing with a preparation method.

In some cases, the implant 10 can be loaded (e.g., manually or using a loader, for example, a loader described in PCT patent publication no. WO 2024/112747, which is incorporated herein by reference for all purposes) into an introducer e.g., a delivery cannula 320 of an implant cartridge 305 of a delivery device 300). Optionally, the introducer can be coupled to a hand piece (e.g., handle) 310 of the delivery device 300 and used to deliver the implant to the target site of the eye.

The terms “biologically-derived material” and “bio-based material” include naturally-occurring biological materials and synthesized biological materials and combinations thereof that are suitable for implantation into the eye. Biologically-derived material includes a material that is a natural biostructure having a biological arrangement naturally found within a mammalian subject including organs or parts of organs formed of tissues, and tissues formed of materials grouped together according to structure and function. Biologically-derived material can include tissues such as corneal, scleral, or cartilaginous tissues as well as acellular biomatrix tissue. Scleral tissue can be especially useful in methods described herein for preparing an ophthalmic tissue implant to be implanted into a target location in an eye (e.g., a ciliary cleft, a supraciliary space, or a portion of Schlemm's canal), for example, because the scleral tissue can provide the implant with appropriate starting structural characteristics useful in such procedures. Biologically-derived material include tissues that are homologous with the tissues of the target implantation site. For example, scleral tissue for implanting against scleral tissue, such as within a subscleral location between the sclera and the choroid. Biologically-derived material includes amniotic membrane. Tissues considered herein can include any of a variety of tissues including muscle, epithelial, connective, and nervous tissues. Biologically-derived material includes tissue harvested from a donor or the patient, organs, parts of organs, and tissues from a subject including a piece of tissue suitable for transplant including an autograft, allograft, and xenograft material. Biologically-derived material includes naturally-occurring biological material including any material naturally found in the body of a mammal. Biologically-derived material as used herein also includes material that is engineered to have a biological arrangement similar to a natural biostructure. For example, the material can be synthesized using in vitro techniques such as by seeding a three-dimensional scaffold or matrix with appropriate cells, engineered or 3D printing material to form a bio-construct suitable for implantation. Biologically-derived material as used herein also includes material that is cell-derived including stem cell(s)-derived material. In some implementations, the biologically-derived material includes an injectable hyaluronate hydrogels or viscomaterials such as GEL-ONE Cross-linked Hyaluronate (Zimmer).

Biologically-derived materials can include naturally-occurring biological tissue including any material naturally found in the body of a mammal that is minimally manipulated or more than minimally manipulated according to FDA guidance under 21 CFR § 1271.3(f) such that the processing of the biological tissue does not alter the relevant biological characteristics of the tissue (see Regulatory Considerations for Human Cells, Tissues, and Cellular and Tissue-Based Products: Minimal Manipulation and Homologous Use, www.fda.gov/regulatory-information/search-fda-guidance-documents/regulatory-considerations-human-cells-tissues-and-cellular-and-tissue-based-products-minimal).

The biologically-derived material, sometimes referred to herein as bio-material, which is used to form the implant can vary and can be, for example, a biologically-derived tissue (sometimes referred to herein as a bio-tissue) including corneal tissue, scleral tissue, amniotic membrane tissue, cartilaginous tissue, collagenous tissue, or other firm biologic tissue. The bio-tissue can be of hydrophilic or hydrophobic nature. The bio-tissue can include or be impregnated with one or more therapeutic agents for additional treatment of an eye disease process.

In some instances, the biologically-derived material can include or be capable of releasing one or more factors of the biologically-derived material for providing additional treatment of a disease or condition. For example, the material can be a tissue that releases healing factors derived from the tissue that have anti-fibrotic, anti-inflammatory, anti-neovascular effects, or the like for repair and regeneration at the site of implantation or near the site of implantation. The material can be whole amniotic membrane that releases one or more regenerative and anti-fibrotic factors that aid in the control of inflammation and scarring including, but not limited to VEGF (Vascular Endothelial Growth Factor), VEGF-R (VEGF Receptor), ANG1 (Angiopoietin 1), TIMP-1 (Collagenase inhibitor), TIMP-2 (Collagenase Inhibitor), IL-1B (Interleukin 1B), PDGF-AA (Platelet Derived Growth Factor), TGFb3 (Transforming Growth Factor Beta 3), bFGF (Basic Fibroblast Growth Factor), and HGF (Heptocyte Growth Factor). The amniotic membrane contains both growth promoting and growth inhibiting proteins (see, e.g., Clinical Ophthalmology 2019:13, 887-894). In some cases, a tissue or population of cells that is capable of releasing one or more regenerative, anti-fibrotic, anti-inflammatory, and/or anti-neovascular factors can be useful as a biologically-derived material for use in forming an implant (e.g., as a result of its structural properties), even if the tissue's or population of cells'ability to release the one or more factors is reduced or destroyed by exposure to a chemical or agent of one or more steps of a method disclosed herein.

The amniotic membrane tissue can be derived from amniotic sac of the placenta. The tissue can be freeze-dried, lyophilized membrane or otherwise minimally manipulated prior to implantation (see, e.g., SURGRAFT dehydrated amniotic sheets, or SURSIGHT ocular amniotic membrane allograft, Surgenex Scottsdale, AZ).

The amniotic membrane can be used as an adjunct treatment with a bio-tissue implant. The amniotic membrane can also be used alone as a primary treatment. The tissue implant alone or with amniotic membrane provided as an adjunct treatment can be implanted in any of a variety of locations including the suprachoroidal space, supraciliary space, Schlemm's canal, cornea, anterior chamber, posterior chamber, intravitreal, epiretinal, subretinal, or other part of the eye.

The tissue implant material can be used in combination with one or more therapeutic agents such that it can be used to additionally deliver the agent to the eye. In an implementation, the bio-tissue can be embedded with slow-release pellets or soaked in a therapeutic agent for slow-release delivery to the target tissue.

Non-biologic material includes chemically synthesized or synthetic materials prepared through artificial synthesis, processing, or manufacture that may be biologically compatible, but that are not cell-based or tissue-based. For example, non-biologic material includes synthetic polymers, copolymers, polymer blends, and plastics. Non-biologic material includes inorganic polymers such as silicone rubber, polysiloxanes, polysilanes, and organic polymers such as polyethylene, polypropylene, polyvinyls, polyimide, etc. Non-biologic material can also include metals, such as Nitinol, stainless steel, titanium, and other metals. Non-biologic materials may be chemically synthesized in a lab and not grown or isolated from a biological source.

The bio-based tissue implant(s) implanted in the eye may have a structure and/or permeability that allows for aqueous outflow from the anterior chamber when positioned in the eye (e.g., within a cyclodialysis cleft, the supraciliary space, suprachoroidal space, Schlemm's canal, trabecular meshwork, and the like). The biologically-derived tissue can be minimally-modified or minimally-manipulated tissue for use in the eye, for example, wherein the biologically-derived tissue has been prepared according to a preservation method and/or trephined to a shape. The minimally modified biologically-derived tissue does not have a systemic effect once implanted and is not dependent upon the metabolic activity of any living cells for its primary function. The biologically-derived tissue can be minimally-manipulated during each step of the method of preparation and use so that the original relevant characteristics of the biologic tissue are maintained. The tissue implant can be a structural tissue that physically supports or serves as a barrier or conduit, for example, by maintaining at least in part a ciliary cleft formed in the eye and/or by positioning at least in part within Schlemm's Canal to aid in outflow through the trabecular meshwork.

At least one implant can be positioned inside the eye in an implanted location such that at least a first portion of the implant is positioned in or flush with the anterior chamber AC and a second portion of the implant is positioned within tissues such as within the supraciliary space and/or suprachoroidal space of the eye. Alternatively, at least one implant can be positioned such that at least a portion of the implant is positioned within Schlemm's Canal and a second portion of the implant is positioned against and/or within the trabecular meshwork and/or the anterior chamber of the eye (see FIGS. 1, 2A-2D). The implant can be sized and shaped such that the implant can be positioned in such a configuration. The implant can be sized and shape so that upon positioning in a first location in the eye, the implant rehydrates and expands into another location in the eye. The implant can provide or otherwise serve as a passageway for the flow of aqueous humor away from the anterior chamber AC (e.g., to the supraciliary space and/or suprachoroidal space, into Schlemm's Canal). In FIGS. 1, 2A-2D, FIGS. 4D-4E, and 5D, the implant 10 is represented schematically as an elongated body or short plug relative to a delivery cannula 320. The size and shape of the implants described herein can vary. Additionally, the size and shape of the implant prior to insertion within the delivery cannula 320 can change upon insertion into the delivery cannula 320 and/or can change after deployment from the delivery cannula 320, which can include rehydration of the implant and expansion to a larger size.

More than one implant 10 can be positioned within the eye during a procedure to improve outflow of aqueous from the anterior chamber. For example, one or more pairs of implants 10 can be introduced and positioned adjacent to one another to form a longitudinal gap between them. The pairs of implants 10 form a scaffold that props open tissue of the eye creating a lake or reinforced drainage reservoir of aqueous within the longitudinal gap. The reinforced drainage reservoir formed by multiple implants can provide additional advantage over a single implant that stents the ciliary body without forming a significant reservoir adjacent the implant. One or more implants 10 can be introduced within Schlemm's Canal as will be described in more detail below (see FIGS. 2A-2B). The implant 10 is preferably implanted ab interno, for example, through a clear corneal incision although ab externo via a scleral incision is possible as well. The implant can be deployed to create an opening or cleft for augmented outflow communication from the anterior chamber AC, such as between the anterior chamber AC and the supraciliary space, the anterior chamber AC and the suprachoroidal space, the anterior chamber AC and Schlemm's Canal, and/or the anterior chamber AC and the sub-conjunctival space, or any other ocular compartment, tissue or interface where trans-scleral, sub-scleral, or supra-scleral occlusion, stenting, and/or tissue reinforcing are clinically indicated. In a preferred implementation, the implant 10 is implanted such that a distal end is positioned within a supraciliary position and the proximal end is positioned flush with or at least partially within the anterior chamber AC to provide a supraciliary cleft. In other implementations, the implant 10 is implanted transmurally across Schlemm's Canal such that a distal end is positioned within the canal and the proximal end is positioned through the trabecular meshwork, including flush with or at least partially within the anterior chamber AC. The distal end of the implant 10 can be positioned between other anatomical parts of the eye, such as circumferentially within Schlemm's Canal without any portion of the implant 10 being left within the anterior chamber AC. In still other implementations, the implant 10 is implanted circumferentially within Schlemm's Canal such that at least a portion of the implant 10 is positioned within the canal and at least another portion of the implant 10 is positioned at least partially outside the canal, for example, within or through the trabecular meshwork or so that at least a portion of the implant 10 extends within the anterior chamber AC.

Glaucoma stenting/shunting devices are typically formed of non-biological materials such as polyimide, metal, or other synthetic non-biological materials that can cause endothelial tissue damage leading to progressive, long-term, and irreversible corneal endothelial loss. The biomaterials (e.g., tissue) of the implants described herein can reduce and/or eliminate these risks of tissue damage while still providing enhanced aqueous humor outflow.

A biologically-derived tissue of the implant 10 can but need not be incorporated into the eye's inherent anatomy after placement in the eye. In some implementations, the tissue implant can be partially or completely absorbed into the host tissue surrounding the tissue implant at the implantation location. In particular, functional incorporation of a tissue implant into a target location of a subject's eye (e.g., a ciliary cleft, a supraciliary space, or a portion of Schlemm's canal) can be facilitated by using an allogenic biologically-derived tissue, especially if the allogenic tissue is derived from the same tissue (i.e., homologous tissue) as the target location in the subject's eye (e.g., an implant formed from an allogenic scleral tissue sample to be used in a target location comprising scleral tissue). The implant 10 can cause the surrounding tissue to form a pathway that remains open for an extended period, even after absorption of the implant material. In some implementations, the biologically-derived tissue of the tissue implant may not significantly absorb or be incorporated into the eye's anatomy such that the implant 10 remains implanted for an extended period of time or indefinitely, as needed.

The biologically-derived tissue need not originate from the eye. For example, the biologically-derived tissue can be amniotic membrane. In some embodiments, an implant described herein formed from amniotic membrane can be useful, e.g., because amniotic membrane can be a naturally thin material and/or because of the optical clarity of amniotic membrane. The amniotic sac surrounding a fetus includes the amnion and chorion, both derived from the inner layer of the placenta. The amniotic membrane is part of the amnion and includes a layer of epithelial cells, a basement membrane, and an avascular stromal matrix. Amniotic membrane contains pluripotent cells, highly organized collagen, anti-fibrotic and anti-inflammatory cytokines, immune-modulators, growth factors, and matrix proteins and can promote healing in eyes (Murri et. al. Clin. Ophthalmol. (2018) 12:1105-1112). While implanted, the amniotic membrane can act as a spacer or tissue reinforcer or adjunct to another structural element (e.g., scleral tissue implant) while releasing factors useful for tissue repair and regeneration. The amniotic membrane bio-absorbs from at least about 3 to 4 weeks up to about 3 to 6 months. After being absorbed, a structural footprint or reservoir or channel remains in the implantation location. That structural footprint can be in the suprachoroidal space, supraciliary space, or Schlemm's canal, which can aid in improving aqueous outflow from the eye. The structural footprint can be created in other locations for other ocular treatments, such as in the anterior chamber, anterior angle, posterior chamber, intravitreal, epiretinal, subretinal, corneal, scleral, or other sites to treat a variety of other eye conditions, such as uveitis, endothelial loss, intraocular inflammation, etc. Thus, implants formed of amniotic membrane can be used to create a semi-permanent spacer and also be used for delivery of beneficial diffusible factors from the tissue that can be helpful against a variety of conditions. The implants formed of amniotic membrane can be implanted alone as a primary treatment or in combination with another tissue implant(s) formed of other materials (e.g., sclera) as an adjunct anti-fibrotic or anti-inflammatory treatment with temporary scaffolding, which will be described in more detail below. Where the tissue implant is described herein as being derived from scleral or another ocular tissue, amniotic membrane tissue is also considered.

As used herein, terms like stent, biostent, implant, shunt, biotissue, or tissue are not intended to be limiting to any one structure or material, aside from being formed of biologically-derived material, preferably tissue, such as scleral tissue or corneal tissue.

The structure implanted can but need not be a material that is absorbed substantially into the eye tissue after placement in the eye such that, once absorbed, a space may remain where the structure was previously located. The structure once implanted may also remain in place for an extended period and not substantially erode or absorb. In some cases, the structure can be permanently implanted at the implantation site.

As mentioned, the biologically-derived material can have a permeability or porosity that allows for aqueous filtration for sufficient control or regulation of intraocular pressure. Permeable bio-tissues described herein (e.g., scleral tissue, corneal tissue, amniotic membrane, etc.) are preferred materials. Any bio-tissue, even if impermeable, is considered herein as a potential material to serve as a structural spacer that keeps a tissue space, such as a cyclodialysis or an opening through the trabecular meshwork, open. Preferably, the material of the implant can create a gap that allows fluid to flow. The gap created can run longitudinally along each side of the implant. Where one or more pairs of implants are introduced so that each implant of the pairs of implants are positioned adjacent to one another to form a longitudinal gap between them, the gap created that runs longitudinally along each side of the implant can be widened into a lake or reservoir of aqueous. The pairs of implants form a scaffold that props open tissue of the eye between the implants that is wider than can be achieved by a single implant. For example, a second implant can be positioned at least an implant width away from the first implant to create a corresponding sized longitudinal gap to act as an aqueous filtration reservoir. The implants can be positioned at least about 50 microns up to about 3,000 microns away from one another to maintain a scaffold of the tissue between the implants. If the material of the implant is permeable, more fluid can pass through the target implantation site (e.g., the cyclodialysis or Schlemm's Canal) than if the material is impermeable and the fluid passes along the outside of the implant. Thus, the material considered herein need not be porous to provide the desired function. The function can be enhanced by the porosity of the material.

Generally, the biologically-derived tissue has some firmness and intraocular durability such that it can maintain outflow from the anterior chamber and is less stiff than non-biologically-derived shunts used in the treatment of glaucoma (e.g., CYPASS, Alcon). The tissue may have a sufficient structure to serve as a spacer to prop open a space for sustained outflow. The tissue can maintain its structural height or thickness once implanted within the target location (e.g., cyclodialysis or Schlemm's canal) such that fluid flow through or around the implant is provided. In some implementations, the cut implant is minimally manipulated by dehydrating, physically compressing, or compacting the implant (for example, to decrease one or more of a length, width, and/or thickness of the implant) before introducing the implant into a delivery shaft so as to alter the size and/or shape of the implant from a first size into a second, smaller size that is more easily placed within the shaft. In some implementations, the implant can be cut from tissue (e.g., a scleral or corneal tissue sample) that is dehydrated. The implant can have a smaller size while dehydrated that increases by at least about 50%, at least about 100%, at least about 200%, up to about 215%, up to about 250%, up to about 300%, up to about 400%, up to about 460%, up to about 500%, or more than 500% following hydration, such as upon deployment in the eye. In some cases, full hydration or full rehydration of a tissue implant that has been prepared can be completed in less than about 1 minute, less than about 2 minutes, less than about 3 minutes, less than about 4 minutes, less than about 5 minutes, from 1 minute to 5 minutes, from about 5 minutes to about 10 minutes, from about 10 minutes, to about 15 minutes, from about 15 minutes to about 20 minutes, from about 20 minutes to about 25 minutes, from about 25 minutes to about 30 minutes, or from about 15 minutes to about 30 minutes.

The delivery cannula can be sized and shaped to be inserted through a cornea (such as a self-sealing incision in a cornea) into the anterior chamber and advanced towards the iridocorneal angle. The delivery cannula can deploy the implant between tissue layers near the angle, for example, within the suprachoroidal/supraciliary space or through the Trabecular Meshwork into Schlemm's Canal. Once the implant is deployed from the delivery cannula, if compacted while inside the delivery cannula, it can begin to return towards its original shape and/or size such as by relaxing from the compacted configuration. The implant, if dehydrated while in the delivery cannula, may swell in size once deployed in the eye due to rehydration of the tissue. The cut implant, once implanted, can take on a shape and/or size that is smaller from its original shape and/or size or that is the same as its original shape and/or size. The minimally-modified biological tissue, once implanted, can be used to treat glaucoma by increasing aqueous outflow from the anterior chamber through the target site. Biologically-derived implant material (particularly where the material is homologous tissue relative to the tissues at the target implantation site) provides advantages in terms of biocompatibility, anatomic conformity, and aqueous permeability compared to non-biological materials such as polyimide or metals. Biologically-derived implant material can provide better conformability and compliance to the scleral wall and can be less likely to cause endothelial and scleral erosion/loss over time and with chronic eye rubbing and blinking.

The term “patch” or “patch of material” as used herein refers to a piece of biologically-derived material having a size along at least one dimension that is greater than a size of the implant cut from the patch of material and implanted in the subject. In some implementations, the patch of material can have a first shape and the implant cut or trephined from the patch of material can have a second, different shape. For example, the patch of material can be about 7 mm (millimeters) wide×7 mm long×0.55 mm thick and the implant cut from the patch of material can be 0.3-1.0 mm wide×7 mm long×0.55 mm thick. The dimensions of the patch of material and the cut implant can vary. The patch of material prior to cutting can be between about 5 mm to about 10 mm wide, between about 5 mm up to about 10 mm long, and between about 0.25 mm to about 2 mm thick, preferably about 0.4 mm-0.7 mm thick or about 0.4 mm to about 1.2 mm thick. The implant cut from the patch of material can be between about 0.3 mm up to about 2 mm wide, preferably between 0.7 mm to 1.0 mm wide. The implant cut from the patch of material can be between about 5 mm up to about 10 mm long. The implant cut from the patch of material can be between 0.25 mm to about 2 mm thick. The implant upon implantation and/or upon exposure to a liquid can change from a width of about 550 μm (microns) up to a width of about 1,000 μm. The implants upon deployment in the eye can increase in size by at least 1.2×. In other words, the implant upon deployment can be at least 20% larger (1.2× the original size) compared to the original size prior to deployment. Preferably, the implants increase in size by at least 1.3×, 1.4×, 1.5×, 1.6×, 1.7×, 1.8×, 1.9×, 2× up to about 3× the original size and anywhere in between. The patch of material and the cut implant can each have the same length and the same thickness but differ from one another in width. The patch of material and the implant cut from the patch of material can also have different lengths and thicknesses. For example, the patch of material can have a first thickness and the implant cut from the patch of material have the same thickness, but when implanted can swell or be folded or rolled into a different thickness from the patch of material.

The cut implant need not be rectangular in shape and can have a non-rectangular shape such as an angular wedge or any of a variety of shapes to provide a particular clinical result. For example, an implant cut to the shape of a “dog bone” having enlarged distal and proximal ends may provide additional fixation within the target tissues. The implant can be cut to have a narrow elongate shape on a leading end and an enlarged dimension on a trailing end to provide ease of insertion as well as at least one end providing fixation.

The form factor of the cut implants can vary as described elsewhere herein. For example, the tissue can be formed into a disk, pellet, plug, bead, or rod shape for implantation in the eye or another location of the body to provide structural support of the target implantation location. The size of the shape can vary depending on the target implantation location. As an example, the implant can be cut from a disk of biologic material having a diameter that is about 5 mm, 8 mm, 10 mm, 11 mm, 12 mm, 14 mm, up to about 15 mm into an implant having a different shape, such as an elongate rod shape that is about 1-7 mm in length and about 0.5-4 mm wide. The implant can have the same thickness as the starting materials (e.g., about 0.05-0.7 mm thick) or can be planed from a first thickness to a second, smaller thickness. The rod shape can be about 3 mm or less in thickness and about 8 mm or less in length as described elsewhere herein. The thickness and the lengths can vary depending on the location of implantation. The implant can be formed of a single tissue type but made into different shapes or dimensions. The different dimensioned implants can be positioned at a target location to achieve a particular combination of structural reinforcement of the target location and, in the case of amniotic membrane tissue, release of healing factors at the site. A combination of tissue types can be implanted together in any of a variety of combinations to provide a desired reinforcement and/or release of healing factors at the target site. For example, amniotic membrane implants and scleral tissue implants can be used in combination.

In some implementations, the patch of material can be a relatively larger width (e.g., 10 mm×10 mm) and the implant cut from the patch to a strip having a much smaller width (e.g., about 1.0 mm to about 1.5 mm) and the cut implant then compacted into a delivery conduit having an inner diameter of about 0.8 mm so that the width of the implant substantially fills the inner diameter. An implant can substantially fill the inner diameter of the delivery conduit even if the implant is not oversized relative to that conduit and thus remains uncompacted. The implant can be oversized relative to the inner dimension of the conduit and be compacted into the conduit to substantially fill it. The implant in its rehydrated state can be oversized relative to the inner dimension of the conduit although in its dehydrated state can be less than the inner dimension of the conduit. This allows for easy sliding of the implant within the conduit prior to and during stages of deployment and maximum stenting following deployment once it rehydrates in the eye. Additionally, the dimension of the cut implant can vary depending on the dimension of the cannula the implant is to be deployed through. For example, the inner diameter of the delivery cannula can be about 300 microns up to about 2800 microns, or about 600 microns to about 800 microns. In some implementations, the delivery cannula is a hypotube having an inner diameter that is less than about 900 microns down to about 228 microns (about 0.009″). The dimensions of the delivery cannula can vary depending on the initial size of the implant and whether the delivery cannula is intended to be delivered ab interno through the cornea or another route of delivery. It may be desired to deliver some implants in an ab externo manner via a scleral flap. In ab externo delivery, the outer diameter of the cannula can be larger compared to a delivery cannula configured to be inserted through a self-sealing corneal incision where minimizing the size is preferred. Where the delivery of the implants is performed ab interno through the anterior chamber via a corneal incision, other approaches are considered as well. Thus, the implant can be cut or trephined to any of a variety of sizes depending on whether the implant is to be compacted into the delivery conduit and depending upon the inner dimension of that delivery conduit.

The implant cut from the patch of material can have a width, a length, and a thickness (sometimes referred to herein as a “height” of the tissue). In an implementation, the width of the implant cut from the patch of material using the cutting devices described herein can be at least 100 microns (micrometers) up to about 1500 microns, or between 100 microns up to 1200 microns, or between 100 microns and 900 microns, or between 300 microns and 600 microns. The implant cut from a patch of material can have a width of at least about 100 microns and a width of no more than 1500 microns, 1400 microns, 1300 microns, 1200 microns, 1100 microns, 1000 microns, 900 microns, no more than 800 microns, no more than 700 microns, no more than 600 microns, no more than 500 microns, no more than 400 microns, no more than 300 microns, or no more than 200 microns. The length of the implant cut from a patch of material can vary depending on the location of implantation. In some implementations, the implant has a length that is between 1 mm and 10 mm, or more preferably between 3 mm and 8 mm long. The thickness of the implant cut from the patch of material can be from 100 microns up to about 1,200 microns, or from 150 microns up to about 600 microns. In an implementation, the biological material forming the implant can have a thickness that is no smaller than 100 microns and no larger than 5 mm. The thickness of the implant can depend upon the particular patch of material being used. The thickness of the implant can also be modified by planning the tissue at the time of modifying. The tissue can thus be cut to have a particular length and a particular width by trephining as well as a particular thickness by planning. The thickness of the implant can also depend on whether the implant is folded or rolled upon implantation such that a patch of material having a thickness of just 250 microns can cut into an implant and the implant folded at implantation to double the thickness to about 500 microns. The thickness of the implant can also depend upon what biologically-derived material is used. For example, scleral tissue or corneal tissue can often have a thickness of around 600 microns, but following harvest can shrink to about 250-400 microns. As such, an implant cut from a shrunken patch of corneal tissue may have a thickness of just 250 microns. An implant cut from a dehydrated sclera or cornea, or other tissue may have a maximum thickness of about 600 μm (microns) and a hydrated maximum thickness of about 1000 μm (microns).

The dimensions of the implant in the dehydrated state can vary depending on the target location for deployment. The implant can be cut to have a smaller starting dimension for deployment within Schlemm's Canal compared to deployment within the supraciliary space. For example, where the implant is to be delivered to traverse Schlemm's canal, the implant can be cut to have a width in the dehydrated state that is about 50 microns to about 300 microns. Upon deployment and subsequent hydration of the implant, the width of the implant can increase by at least 5% (to about 52.5-315 microns), at least 10% (to about 55-330 microns), at least 15%, (to about 57.5-345 microns) at least 20% (to about 60 -360 microns), at least 25% (to about 62.5-375 microns), at least 30% (to about 65-390 microns), at least 35% (to about 67.5-405 microns), at least 40%, (to about 70-420 microns), at least 45% (to about 72.5-435 microns), up to about 50% (to about 75-450 microns) of its dehydrated size. Where the implant is to be delivered to another target location, such as the supraciliary space, the implant can be cut to have a slightly larger starting size in the dehydrated state, such as a width that is about 200 microns to about 1,000 microns. Upon deployment and subsequent hydration of the implant, the size can increase by a similar range (e.g., about 5%-50% and anywhere in between).

In some implementations, which is described in more detail below, the implant cut from the patch of material is cut to substantially fill the conduit from which it is delivered. In other implementations, the implant can be oversized relative to a dimension of a conduit from which it is deployed. In this implementation, the implant can be cut to have a first size, which is oversized compared to the inner dimension of the delivery conduit. The oversized implant can be primed within the delivery conduit such as by compacting or compressing with a tool so that the implant when primed within the conduit takes on a second, smaller size. In some implementations, biological material used to fabricate the implant can be dehydrated, which can decrease one or more dimensions (e.g., length, width, and/or thickness) of the biological material from a first size to a second, smaller size, for example, to facilitate loading of the implant into the delivery device. The reduction in size resulting from the dehydration can facilitate insertion of the implant into a delivery device, which can reduce the time required to prepare the implant for use. In some embodiments, the biological material can be trephined before dehydration. In some embodiments, the biological material can be trephined after the biological material has been dehydrated. Upon deployment in the eye and release of the implant from the delivery conduit, the implant may achieve a third size approaching its original first size. The third size can be due to the implant that was compacted relaxing back towards its uncompacted size. The third size can be due to the implant that was dehydrated within the delivery cannula being rehydrated by the bodily fluids once deployed within the eye.

In a non-limiting example, the tissue implant has dimensions no smaller than 0.1 mm and no larger than 8 mm in any direction and a thickness of not smaller than 50 microns and not larger than 8 mm. In a non-limiting example, the tissue implant is about 6 mm in length by 300-600 microns wide by 150-600 microns thick. The cutting can be no smaller than 1 mm and no larger than 8 mm in any direction. In a non-limiting example, the cut tissue has dimensions of 100-800 microns in width and 1 mm- 10 mm in length. Multiple implants may be delivered to one or more target locations during an implantation procedure.

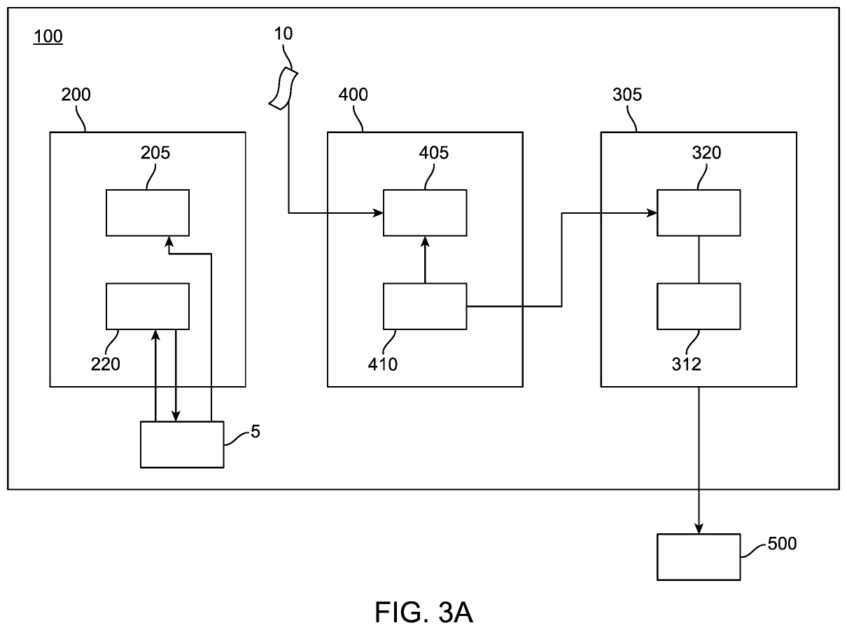

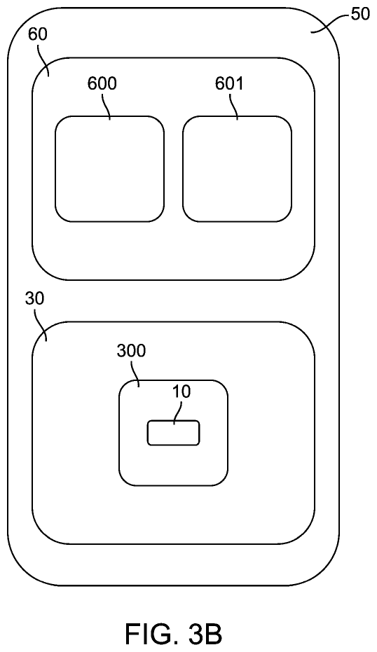

FIG. 3A is a box diagram showing an implementation of a tissue system 100 including one or more of a trephination system 200, an implant cartridge 305, and a loading system 400. The system 100 can additionally incorporate a bio-tissue material 5 such as the tissues described elsewhere herein that are harvested such as from a donor and configured to be shaped such as by the trephination system 200 for formation into an implant 10. The implant 10 from the bio-tissue material 5 can be loaded into a cartridge that can be packaged for storage and transport using the tissue system 100 of FIG. 3A.

Again, with respect to FIG. 3A, the trephination system 200 can include a trephine 205. The trephine 205 of the trephination system 200 is configured for excision modification and shaping of a bio-tissue material 5 into an implant 10 for ocular implantation. The trephination system 200 can further incorporate a plane 220 configured to modify a height or thickness of the bio-tissue material 5. The trephination system 200 is optional. The system 100 need not incorporate a trephination system 200 per se and can rely on other methods of shaping the bio-tissue material 5 into the desired shape of an implant 10.

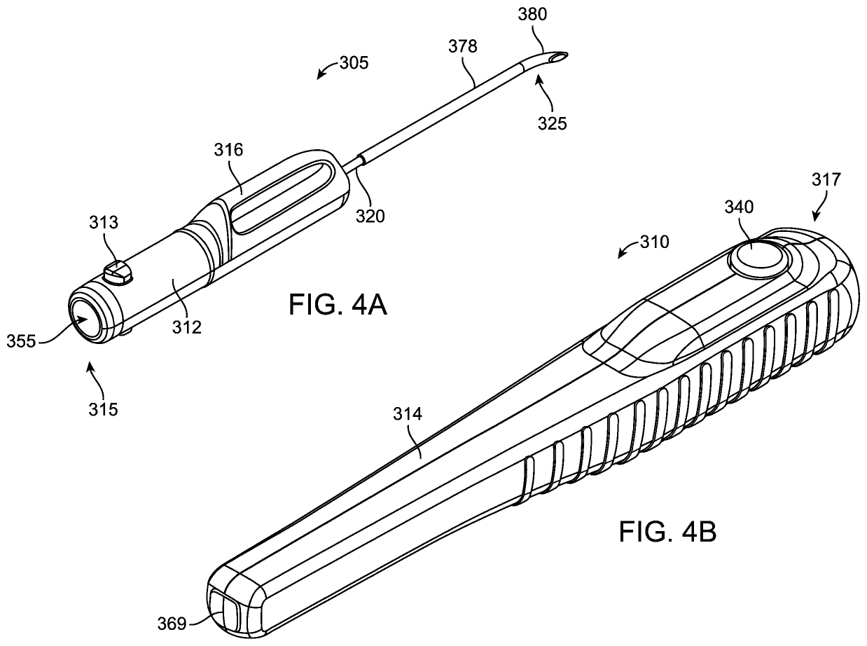

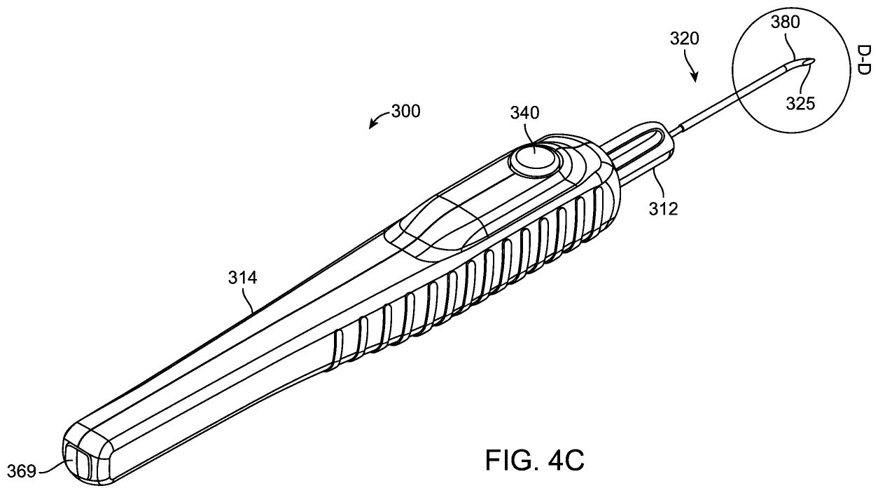

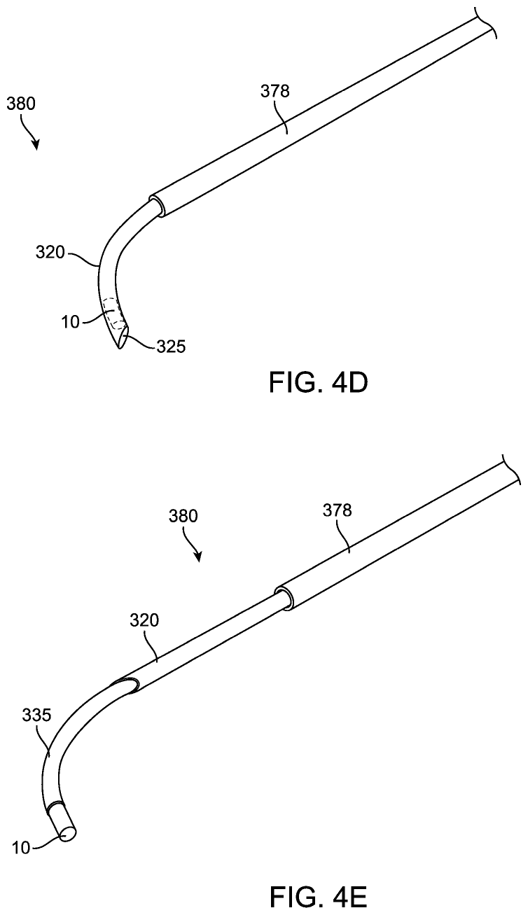

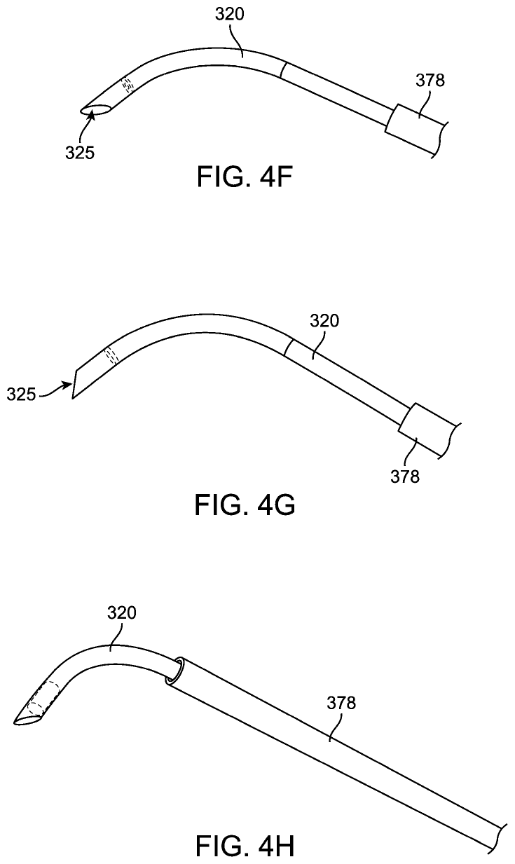

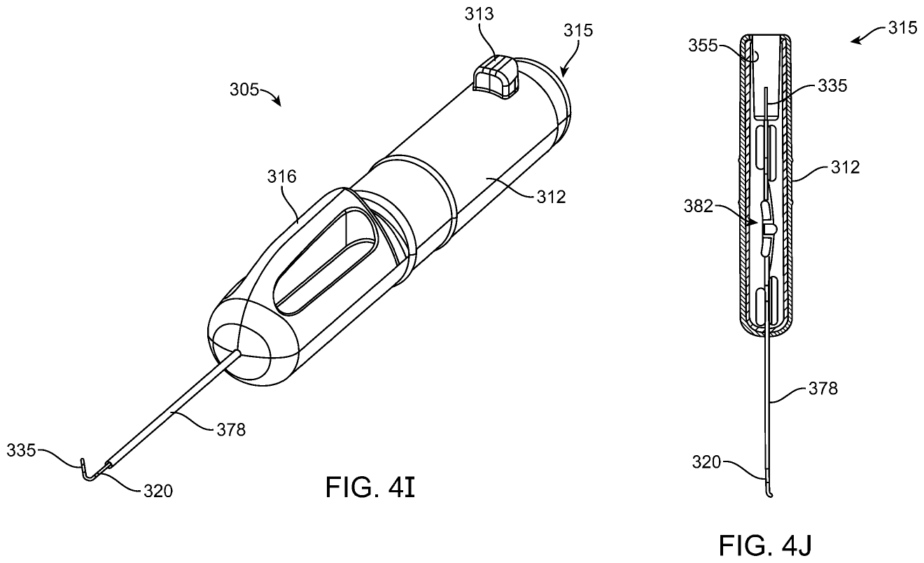

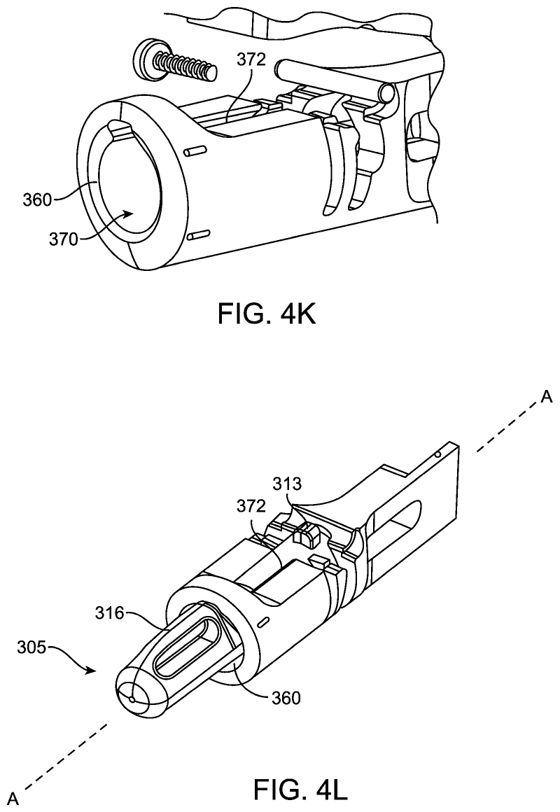

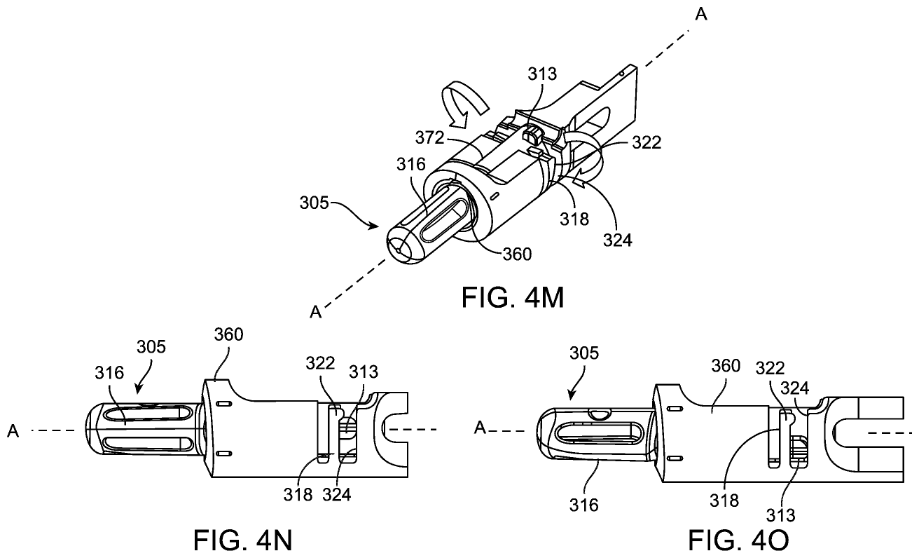

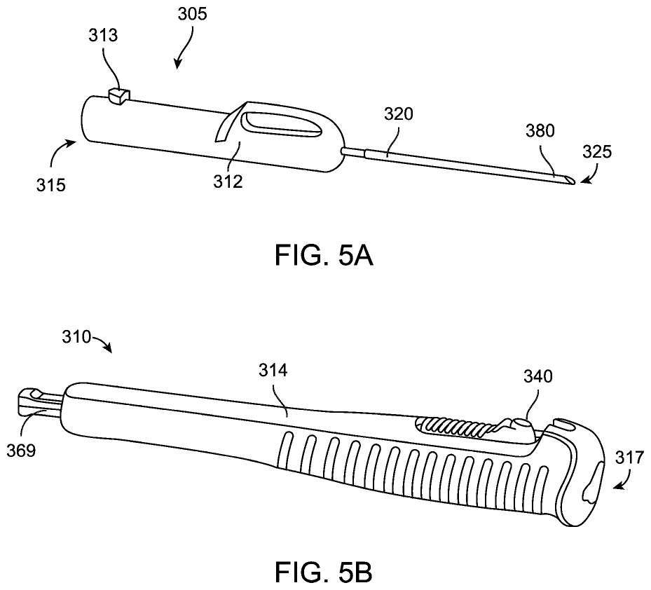

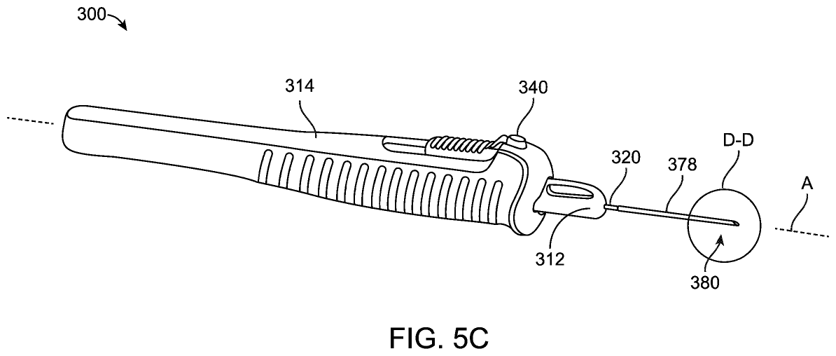

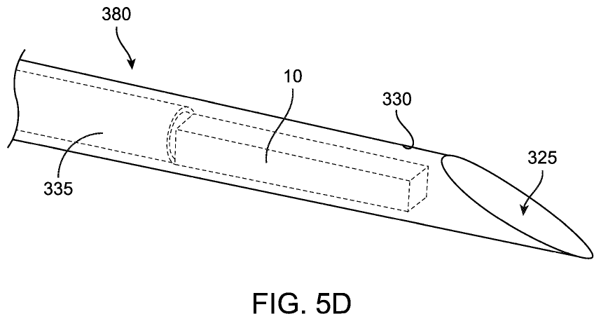

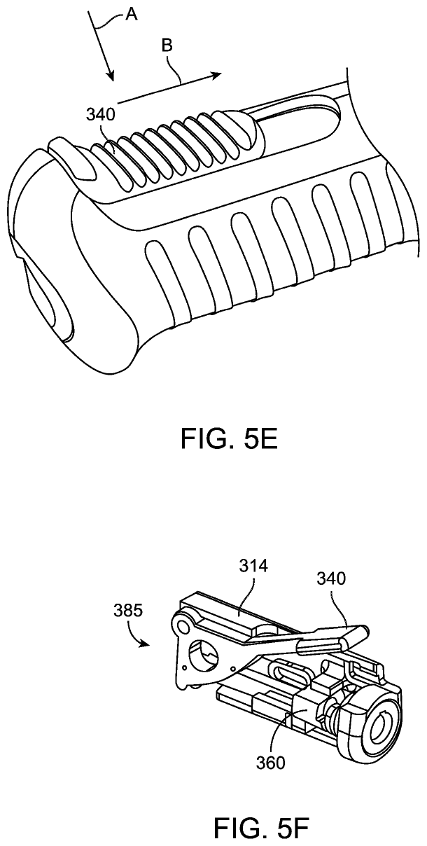

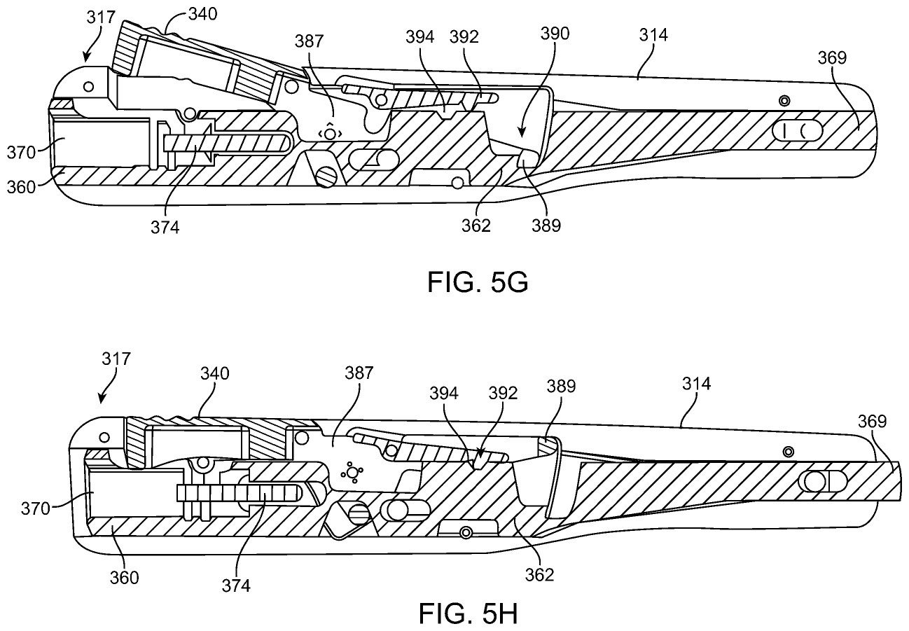

The implant cartridge 305 can include a body 312 having a delivery cannula 320 on a distal end and a proximal end region 315. The proximal end region 315 of the body 312 is configured to couple to a hand piece 310 (including any of the hand pieces 310 described herein and shown in FIGS. 4B, 5B, 5E, 5G, 6A, 6D). The delivery cannula 320 projecting distally from the body 312 is configured to insert within an eye of a patient so that at least a distal end region of the delivery cannula 320 can insert within a treatment location. The delivery cannula 320 has a lumen 330 (see FIG. 5D) and a distal opening 325 into the lumen 330. The implant cartridge 305 is configured to hold the implant 10, such as within the lumen 330 of the delivery cannula 320 for storage and transport such as from a manufacturing facility to a treatment facility such as an operating room.

Still with respect to FIG. 3A, the loading system 400 can include a loading cartridge 405 configured to engage with a loader 410 for hydraulically loading the cut implant 10 into the implant cartridge 305. In some embodiments, the implant 10 can be suspended in liquid within the loading cartridge 405 of the loading system 400 and is transferred from the loading cartridge 405 into the delivery cannula 320 using the loader 410 to inject (or aspirate) the implant 10 into the delivery cannula 320. The loader 410 can inject the implant 10 apply positive pressure to or aspirate the implant 10 using negative pressure through the distal opening 325 of the cannula lumen 330. The loading system 400 is optional. The system 100 need not incorporate a loading system 400 per se and can rely instead on loading the implant 10 into the cannula 320 using micro-forceps and/or other tools to ensure the implant 10 is positioned within the lumen 330.

The implant cartridge 305 loaded with the implant 10 within the delivery cannula 320 can be detached from the loading system 400 and packaged for storage and transport, for example, to a treatment facility such as an operating room where it is used in a surgical procedure upon coupling with a delivery hand piece. The implant cartridge 305 can be contained within packaging 500 including one or more specific features intended to prevent the implant 10 contained within the implant cartridge 305 from being damaged or from separating from the implant cartridge 305, including one or more physical barriers configured to maintain a wet implant 10 wet or a dry implant 10 dry during shelf life and/or to prevent the implant 10 from becoming dislodged from the cartridge 305 within the packaging 500. Storing the implant 10 (e.g., dehydrated) can be contained within the packaging without liquid can be advantageous, as there is no risk of liquid evaporating through the sterile packaging, increasing the length of time an implant can be stored in the container. The packaging 500 can be a radiation stable container.

At the time of surgery, the implant cartridge 305 having the implant 10 already loaded within the delivery cannula 320 is reversibly attached to the proximal hand piece 310 via a coupling feature on a proximal end region 315 of the implant cartridge 305 that is configured to engage a corresponding coupler on a distal end region 317 of the proximal hand piece 310 thereby forming the delivery device 300. The coupling feature(s) on the implant cartridge 305 can include one or more of a luer region 355 on the proximal end region 315 of the body of the cartridge or one or more alignment features, such as projections 313, 316 as shown in FIGS. 4A, 4I-4J, 4L-4O, 5A, 5C. The corresponding coupling feature on the hand piece 310 can include an internal carriage 360 having one or more alignment features 372 as shown in FIGS. 4K-4O, 5G-5H, 6B-6C. Each will be described in more detail below. To prevent premature deployment of a pre-loaded implant 10 upon coupling the implant cartridge 305 with the hand piece 310, a safety feature can be incorporated into the hand piece 310, such as a grenade pin or other mechanism. The delivery device 300 is used to deploy the pre-loaded implant 10 in the eye for anatomic reinforcement, stenting, occlusion, interdigitation, and repair in aqueous drainage procedures. The pre-loaded implant is optional for the implant cartridge 305. For example, the implant cartridge 305 can be loaded with the implant 10 at the time of surgery.

In some implementations, the target site to receive the implant 10 can be pre-dilated or can be dilated by the act of implanting the tissue. As an example, Schlemm's canal can be pre-dilated with a tool inserted within a region of the canal prior to delivery of the implant. As another example, Schlemm's canal can be dilated because of the implant positioned in the canal. Inserted tissue can cause dilation of the canal, which, in turn, can expand and/or dilate the trabecular meshwork thereby improving aqueous flow and drainage to reduce intraocular pressure. As another example, Schlemm's canal can be dilated because of a fluid being delivered within a region of the canal prior to delivery of the implant. Schlemm's canal can be dilated due to a combination of one or more of a tool, the implant, and/or the fluid dilation.

FIG. 3B is a box diagram showing an implementation of a treatment system 50 including one or more of a dilation system 60 and an implant delivery system 30. The dilation system 60 can include a dilation tool 600 and, optionally, a fluid delivery device 601. The implant delivery system 30 can include an implant 10 held within a delivery device 300 used to deploy the implant 10. Each will be described in more detail herein.

Again with respect to FIG. 1, which illustrates schematically a dilated segment 7 of Schlemm's Canal 2 with an implant 10. The implant 10 can be generally oversized relative to the diameter of the canal 2 forming a dilated canal segment 7. In some implementations, the implant 10 can become oversized by rehydrating upon implantation within the canal 2 thereby dilating the canal 2. In other implementations, the implant 10 is oversized or becomes oversized, but is positioned within a canal 2 pre-dilated by another tool or method unrelated to implantation of the tissue. As such, the dilated canal segment 7 of FIG. 1 may be formed by a separate tool that is sized to stretch and expand the segment of Schlemm's Canal 2 without tearing the Trabecular Meshwork 8 so that it remains intact along the segment 7, as described in more detail below.

In some implementations, a single tissue implant 10 is sufficient to provide the homogenous scaffolding desired to provide aqueous outflow, such as through Schlemm's Canal 2 or another region of the eye (e.g., cyclodialysis cleft). Multiple implants 10 can be inserted, such as 2, 3, 4, 5 or more implants 10, within a target site, such as through Schlemm's Canal 2 or another region of the eye (e.g., cyclodialysis cleft), to provide scaffolding for aqueous outflow. The multiple implants 10 can be inserted within a target site and separated from one another so they are not in physical contact with one another upon deployment, such as illustrated in FIGS. 2A-2B. The multiple implants 10 can be inserted within a target site so as to be in direct contact with one another. The multiple implants 10 can be inserted as a stack so their short sides are aligned and positioned substantially end-to-end within the target site (and optionally in contact with one another). When inserted end to end within a target site, the arc length of implant insertion is the sum of each implant's length plus any space between them, if present. The multiple implants 10 can be inserted as a stack positioned so that their elongate sides overlap one another and their short sides are aligned within a single plane. When inserted in this manner within a target site, the arc length of implant insertion is equal to one implant's length and the height or thickness of the implant insertion is equal to the sum of each implant's height or thickness plus any space between them, if present. The multiple implants 10 can be inserted as a stack positioned so that their elongate sides align with one another and the short sides are aligned within a single plane. When inserted in this manner within a target site, the arc length of the implant insertion is equal to one implant's length, the height or thickness of the implant insertion is equal to one implant's height or thickness, and the width of the implant insertion is equal to the sum of each implant's width plus any space between them, if present.

FIG. 2A shows two implants 10a, 10b positioned a short distance away from one another within a dilated canal segment 7. The trabecular meshwork 8 spans the dilated canal segment 7 between the implants 10a, 10b. The distance between the implants 10a, 10b can vary, but is generally close enough so create and/or maintain the dilated canal segment 7 to improve aqueous outflow and drainage. The multiple implants 10a, 10b can be inserted to segregate one segment 7 of Schlemm's Canal 2 from the rest of the canal's circumference. This segregated segment 7 can have its trabecular meshwork 8 cut or pierced or removed without disrupting the function or flow mechanics of the trabecular meshwork 8 of other sections of the canal 2 outside the segregated section (see FIG. 2B). The tissue for the Schlemm's canal implants 10a, 10b are preferably dehydrated scleral tissue, but can be any of a variety of tissues described herein. The dehydrated scleral tissue can be rehydrated to cause the tissue to expand and further dilate the canal in the section. FIGS. 2C and 2D illustrate in schematic example delivery devices 300 for deploying the Schlemm's Canal implants 10a, 10b. The delivery device 300 can include a curved cannula (FIG. 2C) or a straight cannula (FIG. 2D). The second implant 10b located within the delivery device 300 is illustrated as having a smaller form than the implant 10a already in position within the dilated canal segment 7 to represent the dehydrated tissue inside the cannula having a smaller dimension compared to the dimension of the rehydrated implant 10b within the dilated canal segment 7. The implants can be inserted into the canal 2 in a sideways motion using the curved cannula of FIG. 2C or can be inserted directly into the canal 2 in an axial motion with the straight cannula of FIG. 2D.

For manufacturing the implant, a donor globe received at a tissue bank can be used to harvest a patch of material having a particular width, length, and thickness. The patch of material 5 can be scleral tissue that is harvested about 5-10 mm from the limbus towards the posterior pole. The patch of material 5 can be cut into a shape, such as a disk, square, rectangle, or ring of material. The patch of material can be freeze-dried or remain wet. In some implementations, thickness (or tissue height) of the patch of material 5 is modified by planing. Optionally, the starting thickness can be measured before the patch of material 5 is planed down to a desired thickness. The measurements can be performed using a pachymetry device or using a mechanical or electronic thickness gauge. The patch of material 5 need not be planed nor does the thickness need to be measured. Preferably, the thickness is within about 50 microns of the inner diameter of the delivery cannula 320 the implant 10 is to be loaded into. Implants 10 that are too thick will fail a loading step. Thus, to improve yield the patch of material 5 is planed down to about 800 microns, which is a size universally received by most cannulas to be used in a procedure. The patch of material 5 can then be trephined into a strip having a desired width forming the implant 10. The trephine can be a double blade forming a single strip or multiplex blade forming a plurality of strips. The blades can be canted to blade bevel, for example, from about zero degrees to about 25 degrees. The implant 10, which can be an elongated strip of tissue about 5-10 mm in length, can then be loaded into an implant cartridge 305. The implant can be loaded into a cannula 320 of the implant cartridge 305 by aspiration, injection, or physically pushing the implant 10 with a pusher or another tool. The implant cartridge 305 loaded with the implant 10 can then be packaged for storage and transportation. For example, the implant cartridge can be packaged into a moisture barrier pouch or tray. If the implant is wet, fluids can be added to the packaging to maintain the wet environment. If the implant is dry, the moisture barrier pouch can maintain the dry environment. The pre-loaded implant is optional. For example, the implant 10 can be provided separately packaged from the implant cartridge 305 and loaded manually into the delivery cannula 320 at the time of surgery. One or more of the systems and tools used to trephine, plane, load, and package the implants can be single-use disposable tools or can be autoclaved/cleaned and reused.

Outflow of ocular aqueous humor from the anterior chamber occurs through uveoscleral outflow pathways and through trabecular outflow pathways. In an implementation, one or more implants 10 are implanted within the eye to improve uveoscleral outflow of aqueous from the eye by positioning one or more implants 10 between at least a portion of the supraciliary space and at least a portion of the sclera (e.g., within a cyclodialysis cleft). The implant(s) 10 is positioned to create an opening or cleft for augmented outflow communication between the anterior chamber and the supraciliary space and/or the suprachoroidal space. In a preferred implementation, one or more implants 10 processed according to the methods described herein are implanted within the eye to improve trabecular outflow of aqueous from the eye by positioning one or more implants 10 through the trabecular meshwork and within at least a portion of Schlemm's canal. The implant(s) 10 is positioned to create an opening or cleft for augmented outflow communication between the anterior chamber and Schlemm's Canal.

The target locations described herein are extremely small or microsized (e.g., Schlemm's Canal is about 100 microns inner diameter). However, delivering implants formed of tissue using minimally-invasive, ab interno implantation procedures is challenging. For example, tissue can wrinkle within the small sized cannula during loading and deployment steps to these microsized locations. The processing methods described herein dehydrate the tissues into a tissue form that can be cut precisely into sizes suitable for ab interno insertion within the microsized eye anatomy without the issues hydrated or fresh tissue pose. The processed tissue provides microsized implants have a resulting tissue form that provides both deliverability and sufficient scaffolding support within the target anatomy.

Specifics of the delivery devices 300 and components in the use thereof for implantation into the trabecular outflow pathway and/or the uveoscleral outflow pathway are shown in FIGS. 4A-4O and FIGS. 5A-5H, FIGS. 6A-6E, FIGS. 7A-7I.

Each implementation of the delivery device 300 can incorporate an implant cartridge 305 configured to reversibly couple to a proximal housing or hand piece 310. Generally, each of the implant cartridges 305 described herein (including the implant cartridges of FIGS. 4A, 4I-4J, 4L-4O, 5A) includes a body 312 coupled to an elongate shaft projecting distally from the body 312. The elongate shaft can be in the form of at least one tubular element or delivery cannula 320. The delivery cannula 320 has a distal end region 380 including a distal opening 325 into a lumen 330 of the cannula 320. The cannula 320 is sized and shaped such that a tissue implant, such as an implant 10 positioned within a distal end region of the of the cannula 320, can be deployed within the eye from the lumen 330 via the distal opening 325 from the lumen 330.

The implants described herein, whether for delivery into uveoscleral outflow pathway or trabecular outflow pathways, can be delivered with a generally straight cannula 320 as shown in FIG. 5A, 5C-5D or with a delivery cannula 320 having a curved distal end region as shown in FIGS. 4A, 4C-4H, 4I-4J. Preferably, for delivery of the implant 10 into the uveoscleral outflow pathway, the cannula 320 can be substantially straight so that the lumen 330 of the cannula 320 has a single longitudinal axis A extending from a proximal end region of the cannula 320 to the distal opening 325 of the cannula 320. Preferably, for delivery of the implant 10 into Schlemm's Canal, the cannula 320 can be curved along the distal end region 380. The lumen 330 of the cannula 320 has a first longitudinal axis along a proximal end region of the delivery cannula 320 and a second longitudinal axis extending through the distal opening 325 of the delivery cannula 320.

At least the distal end region 380 of the cannula 320, whether straight or curved when at rest, is preferably flexible so that the distal end region 380 deflects upon contact with a force. The distal end region 380 is flexible so that it can change configuration dependent on what forces are applied. FIGS. 4A-4C illustrate the distal end region 380 as curved at rest. This can be due to the presence of a curved inner member 335 within its lumen. The inner member 335 will be described in more detail below. The flexible distal end region 380 straightens and aligns coaxial with the longitudinal axis A as the cannula 320 is retracted proximally over the inner member 335 (see FIG. 4E). Alternatively, the flexible distal end region 380 can be straight at rest and arranged so that it deflects to curve away from the longitudinal axis A as the cannula 320 is advanced between tissue layers towards the target delivery site. For example, the inner member 335 can be straight and positioned a distance away from the distal opening. The distal end region 380 can advance between the tissue layers at the anterior angle to create a cyclodialysis. The distal-most end may come into contact with the sclera, which applies a force against the distal end region 380 of the delivery cannula 320 causing the cannula to deflect away from the sclera as the cannula 320 advances along the wall of the eye. The distal-most end of the delivery cannula 320 can be atraumatic so that the cannula 320 does not snag the sclera and instead slides along it.

In some implementations, the delivery cannula 320 includes an outer tube member 378 that forms the distal end region 380 of the delivery cannula 320. The outer tube member 378 can be a clear material, such as reflowed nylon, that makes a continuous smooth channel from a proximal end region of the delivery cannula 320 to the distal end region 380 of the delivery cannula 320. Thus, the delivery cannula 320 can be formed of more than one material, including a first material that is stiffer such as stainless steel or Nitinol along at least a proximal end region of the delivery cannula 320, and a second material that is relatively more flexible, such as Nylon or another polymer. The first material along the proximal end region of the delivery cannula 320 can be opaque and the second material along at least the distal end region of the delivery cannula 320 can be relatively translucent or transparent. The translucent or transparent material allows for a user to visualize the implant 10 located within the lumen 330 of the delivery cannula 320. The distal end region 380 that is formed of the second material that is optionally more flexible and/or optionally translucent or transparent, can be formed to be substantially straight as in the implementation shown in FIGS. 5A, 5C-5D or can be formed into a curve as in the implementation shown in FIGS. 4A-4H and FIGS. 4I-4J. Transitions between the materials can be smooth to prevent the implant 10 within the lumen 330 from becoming misaligned or jammed within the lumen 330 during deployment.

The outer tube member 378, if present, (or the distal end region 380 of the cannula 320 in the implementation if no outer tube member 378) can be curved or straight. Where the distal end region 380 is curved, the proximal end region of the cannula 320 can extend along the longitudinal axis A and the proximal opening into the cannula 320 can surround the longitudinal axis A. The distal end region of the cannula 320 can also extend along the longitudinal axis A and the distal opening 325 from the cannula 320 can surround the longitudinal axis A. The proximal end region of the outer tube member 378 can be coaxial with the distal end region of the cannula 320 whereas the distal end region of the outer tube member 378 can curve or bend away from the longitudinal axis A. Thus, the distal end region of the outer tube member 378 can form a curved distal end region 380 of the shaft. The curved distal end region 380 can be a tangent arc to the proximal end region with radii of between 10-20 mm, preferably about 10-15 mm, or about 12 mm. The distal opening 325, whether formed by the tube member 378 over the cannula 320 or the cannula 320 itself, can be at an oblique angle to increase the size of the distal opening 325. The bevel can be about 10-45 degrees, preferably about 12-16 degrees. The distal-most end of the shaft can form a flat face or designed for blunt dissection. In implementations, the distal end region 380 is straight and formed of a flexible material configured to deflect as the distal end region 380 is advanced between tissue layers towards the supraciliary space and that is at least partially translucent so as to visualize the implant 10 within the lumen 330 (see FIGS. 4D and 5D).

Each implementation of the delivery device 300 can further include an inner member 335 housed at least partially within the lumen 330 of the cannula 320 that aids in deploying the implant 10 from the lumen 330. The inner member 335 can be in the form of an elongate pusher configured to be advanced through the lumen 330 to urge the implant 10 out of the lumen 330 through the distal opening 325. In some cases, the inner member 335 can be in the form of a stopper that stays fixed relative to the hand piece 310 as the cannula 320 is retracted proximally over the inner member 335 (see FIGS. 4E and 4J and also FIG. 5D) to deploy the implant 10 disposed within the lumen 330 and through the distal opening 325. The delivery device 300 can also be configured to perform a combination of pushing the implant 10 through the lumen 330 with a pusher and retracting the cannula 320 over the pusher to deploy the implant 10 from the lumen 330.

The inner member 335 can be straight or can be curved, such as shown in FIG. 4E. The distal end region 380 of the delivery cannula 320 can be sufficiently flexible to take on the shape of the inner member 335 as it is retracted over the inner member 335. Thus, prior to retracting the delivery cannula 320 over the inner member 335, the delivery cannula 320 can have a first shape and after retracting the delivery cannula 320 over the inner member 335, the delivery cannula 320 can have a second shape, that is the same or different from the first shape.

FIG. 4F is a perspective view of a distal end region of a delivery cannula 320 having a beveled distal opening 325. FIG. 4G is a side view of another implementation of a beveled distal opening 325. The inner member 335 positioned internal to the delivery cannula 320 incorporates a distal curvature such that the distal end region 380 of the delivery cannula 320 takes on the shape of the curve. The heel of the bevel in the version shown in FIG. 4F is located on a radially-inward side of the curve whereas the heel of the bevel in the version shown in FIG. 4G is located on the radially-outward side of the curve for a “backwards” bevel direction. FIG. 4H illustrates a cut line to create the backwards bevel. The bevel direction and angle of the bevel of the distal opening 325 can ensure the implant 10 enters and is released cleanly within the canal. The backwards bevel helps to preserve the trabecular meshwork from being inadvertently scraped open by providing a wedge effect into the trabecular meshwork. A flat section of the bevel can be angled against a back wall of the trabecular meshwork and as the bevel advances pries open the meshwork.

Again with respect to FIGS. 4A-4B and also with respect to FIGS. 4I-4J, the body 312 has a proximal end region 315 capable of coupling with a distal end region 317 of the hand piece 310. The proximal end region 315 can have an outer diameter sized to insert within the distal end region of the hand piece 310 into the distal opening 370 of the carriage 360. The proximal end region 315 of implant cartridge 305 can incorporate a coupling feature, such as a luer region 355 configured to couple the implant cartridge 305 with the hand piece 310. FIG. 4J best shows a tapered luer region 355 on the proximal end region 315 of the implant cartridge 305. The inner diameter of the luer region 355 can be tapered. The luer region 355 can also allow for direct connection of a syringe, for example, to load the implant cartridge 305 with the implant using aspiration pressure. The inner diameter of the luer region 355 is sized large enough to receive a buttress 374 fixed within the distal end region of the housing 314 (see FIGS. 5G-5H). As will be described in more detail below, the buttress 374 can be fixed within a region of the housing 314 near the distal opening 370 of the carriage 360 so that it remains in place throughout actuation of the actuation mechanism thereby providing a bearing surface for the proximal end of the inner member 335. The inner member 335 is in turn held fixed as the rest of the implant cartridge 305 including the cannula 320 through which the inner member 335 at least partially extends retracts proximally over the inner member 335 to deploy the implant inside the cannula 320.

The cannula 320, whether substantially straight or having a curved distal end region 380, can incorporate a friction feature 382 near a proximal end region of the cannula 320 that is a curve or bend or combination of curves or bends that redirect the lumen away from the longitudinal axis A of the cannula 320 (see FIG. 4J). The inner member 335 of the delivery system can be native to the implant cartridge 305 and extends at least in part within the lumen 330 of the cannula 320 so that a portion of the inner member 335 spans the curve or otherwise spans the location of the friction feature 382. The friction feature 382 creates friction between the inner member 335 and the inner surface of the cannula 320 at the location of the feature 382 preventing the inner member 335, which is otherwise unattached to the implant cartridge 305. Thus, the inner member 335 can be held within the implant cartridge 305 only by friction against the inner surface of the cannula 320 at the location of the friction feature 382. The friction between the inner member 335 and the cannula 320 can be sufficient to avoid inadvertent movements between the inner member 335 and the cannula 320, for example, during shipping and handling of the implant cartridge 305, until deployment of the implant 10 is desired.

Again with respect to FIGS. 4I-4J, the proximal end region 315 of the implant cartridge 305 can be inserted within the distal opening of the hand piece 310 and into the distal opening 370 of the carriage 360 so that the projection 313, which can include at least a first projection 313 on an upper surface of the proximal end region 315 of the implant cartridge 305. The proximal end region of the inner member 335 can extend outside a proximal end of the cannula 320 of the implant cartridge 305 so that at least a length of the inner member 335 is available within the luer region 355. As discussed elsewhere herein, the distal end region of the inner member 335 can reside within the lumen of the shaft just proximal to the implant 10 (e.g., within the lumen of the outer tube member 378 or within the lumen 330 of the cannula 320) so that at least a length of the inner member 335 traverses the friction feature 382 in the cannula 320. Proximal movement of the cartridge 305 further into the internal bore of the housing 314 causes the proximal end of the inner member 335 within the luer region 355 to abut against the distal end of the buttress 374 fixed within a region of the housing 314. The buttress 374 is held fixed relative to the housing 314 as the carriage 360 is retracted proximally through the internal bore of the housing 314. The inner member 335 can be held within the lumen 330 of the cannula 320 only by friction against the inner surface of the cannula 320 at the curve forming the feature 382. The force of the buttress 374 against the proximal end of the inner member 335 as the implant cartridge 305 and carriage 360 are retracted relative to the housing 314 is greater than the friction between the inner member 335 and the cannula 320 caused by the feature 382. The bearing surface of the buttress 374 abuts the proximal end of the inner member 335 preventing the inner member 335 from moving proximally along with the cannula 320. This results in the inner member 335 traversing the lumen 330 of the cannula 320 as the cannula 320 of the tissue cartridge 305 retracts with the carriage 360 over it, which results in deploying the implant 10 out the distal opening 325.

The friction provided by the feature 382 prevents the inner member 335 from moving inadvertently during shipping and handling of the device that could inadvertently deploy the implant 10. The feature 382 provides a “feel” of deployment upon actuation and as the friction provided by the feature 382 is overcome during retraction of the cartridge relative to the buttress 374 and relative movement of the cannula 320 over the inner member 335.

Still with respect to FIGS. 4I-4J, the proximal end region 315 of the implant cartridge 305 can include at least one proximal alignment feature or projection 313, which can be shaped and sized to prevent the implant cartridge 305 from coupling to the hand piece 310 unless the projection 313 is properly aligned with a corresponding alignment feature located on the distal end region 317 of the hand piece 310 housing 314. The projections 313 can prevent the cartridge 305 from being attached unless the hand piece 310 is in the ready position. The projection 313 can extend from an external surface of the proximal end region 315 of the implant cartridge 305 that is sized and shaped to engage with a corresponding alignment feature(s) 372 on a distal end region of the carriage 360 within the hand piece 310, which will be described in more detail below.

### Loading and Actuation of the Implant Cartridge with the Hand Piece

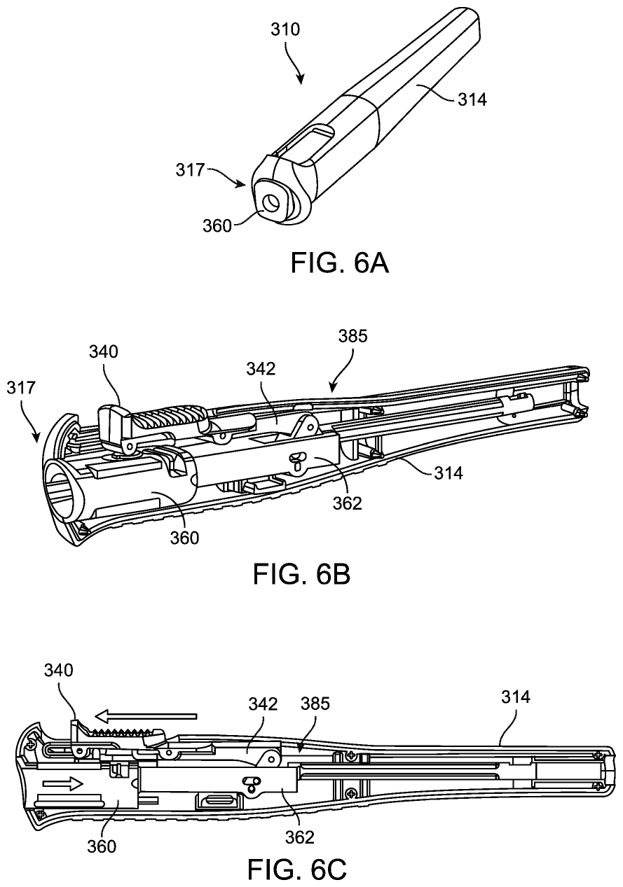

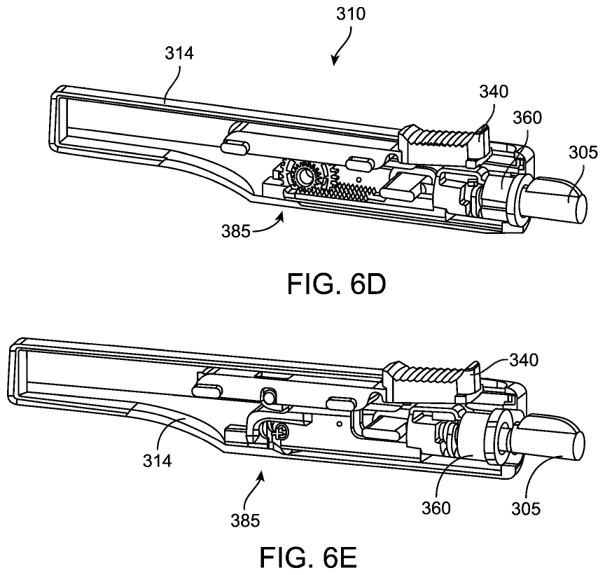

FIG. 4A illustrates an implant cartridge 305 and FIG. 4B illustrates a hand piece 310. The implant cartridge 305 of FIG. 4A and the hand piece 310 of FIG. 4B can couple together forming a delivery device 300. The implant cartridge 305 of FIG. 4A, however, can be used with any of the hand pieces 310 described herein to deploy the implant 10 including the hand piece 310 of FIG. 4B as shown in FIG. 4C, or the hand piece 310 of FIG. 5B, similar to what is shown in FIG. 5C, or the hand piece 310 of FIGS. 5E, 5G-5H, 6A-6C, 6D-6E. Similarly, the implant cartridge 305 of FIG. 5A can be used with any of the hand pieces 310 described including the hand piece 310 of FIG. 5B, as shown in FIG. 5C, the hand piece 310 of FIG. 4B, similar to what is shown in FIG. 4C, or the hand piece 310 of FIGS. 5E, 5G-5H, 6A-6C, 6D-6E. FIGS. 4I-4J illustrate an implant cartridge 305 that also can be used with any of the hand pieces 310 described herein including the hand piece 310 of FIG. 4B, 5B, 5E, 5G-5H, 6A-6C, 6D-6E. Description of a feature with respect to one implant cartridge 305 or hand piece 310 of one of these figures can apply to another implant cartridge 305 or hand piece 310 of one of the other figures. Similarly, description of feature with respect to the cannula 320 or body 312 of one implant cartridge 305 of one of these figures can apply to another implant cartridge 305 of one of the other figures.

The distal end region of the housing 314 is configured to couple with the proximal end region 315 of the implant cartridge 305, such as by a male-to-female attachment mechanism. The distal end region of the housing 314 can define a distal opening into the internal bore of the housing 314 that is sized to receive at least the proximal end region 315 of the body 312. The attachment between the body 312 of the implant cartridge 305 and the housing 314 of the proximal hand piece 310 ensures alignment of the lumen 330 of the delivery cannula 320 with the internal mechanisms of the hand piece 310, such as inner member 335 if it is native to the hand piece 310 or the buttress 374 for the inner member 335 if it is native to the cartridge 305 or any other element or elements that are involved in deployment of the implant 10 from the cartridge 305.

The implant cartridge 305 can be loaded onto the hand piece 310 via a carriage 360. FIG. 4K is a partially exploded detail view of carriage 360 of a hand piece 310. FIGS. 4L-4O illustrate a cartridge 305 (the distal cannula of the cartridge 305 is not shown for simplicity) inserted within the carriage 360 in various states of assembly. The carriage 360 is also visible in FIGS. 5F, 5G-5H and FIG. 6A-6C, 6D-6E. The external dimension of the proximal end region 315 of the cartridge 305 can be sized to insert through a distal opening at an engagement end of the housing 314 to engage with and insert at least partially within the distal opening 370 of the carriage 360, which itself is movable within the housing 314.

When the hand piece 310 is in a ready position and the carriage 360 is advanced to its distal-most position within the housing 314, a user can insert the proximal end region 315 of the cartridge 305 axially into the distal opening of the proximal hand piece 310 and into the distal opening 370 of the carriage 360. When the hand piece 310 is not in the ready position and the carriage 360 is not in its distal-most position within the housing 314, the alignment feature 372 of the carriage 360 is located deep enough within the hand piece 310 to prevent axial engagement and rotational locking of the cartridge 305. The user is prevented from pushing the cartridge 305 deep enough so the projections 313, 372 can engage with one another. The user is prevented from mistakenly attaching the implant cartridge 305 to a hand piece 310 that is not ready to be fired.

FIG. 4K illustrates a portion of the carriage 360 designed to move within the housing 314 of the hand piece 310 and FIG. 4L illustrates the carriage 360 after insertion of the cartridge 305 through the distal opening 370, but prior to locking of the cartridge 305 inside the carriage 360. A proximal projection 313 and A distal projection 316 are positioned on an upper surface of the tissue cartridge 305 and oriented in a 12 o'clock position relative to the longitudinal axis A of the carriage 360 upon insertion through the carriage 360 (see also FIGS. 4A, 5A, and 4I). The proximal projection 313 is visible through an upper surface of the carriage 360 near a rearward end of the alignment feature 172. The distal projection 316 abuts the distal-facing surface of the carriage 360 as the proximal projection 313 bottoms out within the alignment feature 172. Thus, the distal projection 316 is visible distal to the distal opening 370 of the carriage 360, even following full insertion of the cartridge 305 into the carriage 360.

The final attachment between the cartridge 305 and the hand piece 310 can incorporate rotation of the cartridge 305 relative to the carriage 360 (and to the housing 314) to ensure complete coupling, such as via a snap-lock mechanism. The presence of the distal projection 316 outside the carriage 360 and also outside the distal end region of the housing 314 (see FIGS. 4C, 5C, and 6D-6E) provides visual feedback to a user regarding the rotational position of the cartridge 305 within the housing 314.

FIGS. 4L-4O illustrate a snap lock mechanism that provides complete coupling as the cartridge 305 is rotated relative to the carriage 360. As mentioned above, FIG. 4L shows the tissue cartridge 305 oriented in a 12 o'clock position relative to the longitudinal axis A of the carriage 360. FIG. 4M illustrates a first stage of rotation of the cartridge 305 in a clockwise direction around the longitudinal axis A of the carriage 360. The proximal projection 313 rotates towards a snap lock feature formed by a beam 318 having an undercut feature 322 projection within a slot 324. The beam 318 flexes as the proximal projection 313 is urged against its upper surface thereby receiving the proximal projection 313 within the slot 324 of the carriage 360. The beam 318 returns to its upright position upon the proximal projection 313 sliding past the undercut feature 322 thereby capturing the proximal projection 313 within the slot 324. FIG. 4N illustrates the proximal projection 313 rotated about 90 degrees in a clockwise direction into the slot 324 positioned just past the undercut feature 322 and the beam 318 is returned to the upright position. This arrangement provides the complete coupling between the cartridge 305 and the handpiece 310. The rotation to lock the cartridge 305 relative to the carriage 360 can be about 45 degrees to about 90 degrees in either the clockwise or counter-clockwise direction around the longitudinal axis A.

The cartridge 305 can undergo further rotational adjustments after coupling and locking within the carriage 360 of the hand piece 310. The cartridge 305 locked within the carriage 360 of the hand piece 310 can be further rotated relative to the hand piece 310. FIG. 4O illustrates the coupling arrangement can undergo further rotation up to about 120 degrees from the initial insertion position (12 o'clock of FIG. 4L) in either the clockwise or counter-clockwise direction so that the proximal projection 313 approaches a terminal end of the slot 324. For example, the cartridge 305 can be rotated around the longitudinal axis A from 0 degrees (12 o'clock) up to 90 degrees (3 o'clock or 9 o'clock) to achieve locking and further rotation up to about 120 degrees based on a surgeon's preference. Thus, the cartridge 305 can rotate up to about 240 degrees around the longitudinal axis A of the hand piece 310. In use, a scrub tech can prepare the hand piece 310 for a surgeon by inserting and locking the cartridge with the carriage 360 of the hand piece 310 and then rotating the cartridge 305 relative to the hand piece 310 to a desired orientation based on handedness of the surgeon. Any of the cartridges 305 and hand pieces 310 described herein, including those shown in FIGS. 4A-4H, 5A-5H, and FIGS. 6A-6F, can incorporate this rotational adjustment feature.

The position of the distal projection 316 outside the housing 314 can provide the user with visual feedback of the rotation of the cartridge 305 within the carriage 360. The position of the distal projection 316 on the tissue cartridge 305 also can provide visual feedback to a user of the orientation of the distal curve of the inner member 335. The delivery cannula 320 of the cartridge 305 is not shown in FIGS. 4L-4O for simplicity although it would be present in use. FIG. 4I illustrates the tissue cartridge 305 with the delivery cannula 320. As discussed elsewhere herein, the delivery cannula 320 can include an outer tube member 378 and an inner member 335 housed at least partially within the lumen 330 of the cannula 320 that aids in deploying the implant from the lumen 330. The tissue cartridge 305 and the delivery cannula 320 affixed to it can be retracted over the inner member 335. The inner member 335 can incorporate a distal curvature. The orientation of the distal projection 316 on the body 312 of the tissue cartridge 305 alerts a user as to the direction of the curvature of the inner member 335. Any of the cartridges 305 and hand pieces 310 described herein, including those shown in FIGS. 4A-4H, 5A-5H, and FIGS. 6A-6F, can incorporate this feature.

The features on the cartridge 305 (i.e., projections 313, 316, luer region 355, distal curvature of inner member 335) and on the carriage 360 (i.e., alignment feature 372, slot 324, snap-lock features 318, 322, 324) can allow for keyed coupling of the cartridge 305 with the hand piece 310 forming a complete delivery device for use in a patient.

The hand piece 310 includes one or more actuators 340 that cause a tissue implant within the lumen 330 of the delivery cannula 320 to be deployed. In some implementations, the actuation mechanism is configured to retract the implant cartridge 305 and its cannula 320 relative to the housing 314 and the inner member 335 held fixed relative to the housing 314 and the cannula 320, which functionally results in the inner member 335 passively moving through the lumen of the cannula 320 to deploy the implant 10. In other implementations, the inner member 335 is actively moved in a distally direction through the cannula 320 to deploy the implant. In still further implementations, the inner member 335 is actively moved and the cannula 320 is actively moved to achieve deployment of the implant 10. The depth of penetration of the delivery cannula 320 prior to deployment of the implant 10 can vary, but is preferably sufficient to ensure the proximal end of the implant 10 (which is still inside the cannula 320 prior to deployment) is at least between the target tissue layers so that upon retraction of the delivery cannula 320 over the inner member 335, the implant 10 is released within a desired location that is unlikely to inadvertently fall from the distal opening into the anterior chamber.

The motion of the actuator(s) 340 to achieve implant deployment can vary. The motion of the actuator 340 can be a simple push button. The motion of the actuator 340 can be in a push forward (proximal-to-distal direction). The motion of the actuator 340 can be in a distal-to-proximal sliding motion of the actuator 340. The motion of the actuator 340 can incorporate a pivoting motion. Various actuation mechanisms are described below and can be incorporated into each of the hand pieces 310 described herein. Regardless, the specific actuation motion or mechanical feature of the actuator 340, the deployment of the implant from the delivery device creates low to no backlash and maintains the implant cartridge 305 and thus, the cannula 320, within a single plane with low to no rotational forces during actuation of the actuator 340 for deployment.

In some implementations, the hand piece 310 can incorporate an actuator 340 that is spring-loaded to deploy the implant. The spring-loaded deployment actuator 340 has a mechanical advantage allowing for lower push force to actuate retraction. The actuation mechanism can include a spring arming mechanism. The actuation mechanism can include a compression spring that is arranged to urge the carriage 360 proximally through the housing 314. When the actuation mechanism is in the armed configuration, the carriage 360 is in a distal-most position relative to the housing 314, the spring is compressed, and the actuator 340 is ready to be fired to release the carriage 360 to be urged by the spring proximally.

Actuation of the delivery devices described herein can incorporate any of the implementations described in WO 2024/112747, published May 30, 2024.

FIG. 5E is a detailed view of an actuator 340 shown in FIG. 5B that incorporates a two-part activation of deployment. A user can push the actuator 340 inwards or down (arrow A), if the actuator 340 is positioned on an upper side of the housing 314, prior to retracting the actuator 340 proximally (Arrow B) to withdraw the implant cartridge 305. The delivery cannula 320 is affixed to the body 312 of the implant cartridge 305 so that the cannula 320 and the cartridge 305 move together during retraction. The cartridge 305 moves relative to the inner member 335 extending through the lumen 330 of the delivery cannula 320, which remains stationary, causes the implant 10 to be left in position within the desired surgical depth. The inner member 335 is described in more detail with respect to FIGS. 4D-4E and also FIGS. 4I-4J and FIGS. 5G-5H. The actuator 340 of FIG. 5E can be incorporated on any of the hand pieces 310 described herein including the hand piece of FIG. 4B or 5B.

FIG. 5F is a detailed view of an actuator 340 without the remainder of the hand piece 310 being illustrated. The actuator 340 is a push button similar to the push button actuator 340 of FIGS. 5B-5C that retracts the carriage 360 proximally upon depression of the actuator 340 via a pivoting mechanical linkage 385.

FIG. 5G is a cross-sectional view of an actuator 340 similar to that of FIG. 5F. The implant cartridge 305 can engage with the carriage 360 near the distal end region 317 of the housing 314 of the hand piece 310, such as by inserting within the distal opening 370 of the carriage 360. The actuator 340 can be a push button that undergoes one or more steps of actuation to deploy an implant 10 from the implant cartridge 305 (also referred to herein as a carrier). The actuator 340 can be a multi-part actuator 340. The actuator 340 can unlock as a first step and switch into travel mode as a second step. The hand piece 310 can be provided with a safety latch 390 engaged. The safety latch 390 prevents deployment of the implant 10 from the cannula 320 of the cartridge 305 as the cartridge 305 is being loaded within the hand piece 310. In some implementations, the actuator 340 can be in the form of a push button at a distal end region of an elongate lever 387. A mechanical feature 389 can be located on a proximal end region 342 of the lever 387. The mechanical feature 389 is sized and shaped to engage at least a portion of the interior of the hand piece 310, such as a feature near a proximal end region 362 of the carriage 360. Engagement between the feature near the proximal end region 362 and the mechanical feature 389 of the lever 387 forms the safety latch 390. The engagement provides some interference or friction against movement of the carriage 360 in the proximal direction during, for example, insertion of the implant cartridge 305 into the distal opening 370 of the carriage 360.

Still with respect to FIGS. 5G-5H, a user pushes the actuator 340, for example, inwards or down, if the actuator 340 is positioned on an upper side of the housing 314. This movement of the actuator 340 unlocks the safety latch 390 by moving feature 389 out from its engagement with the feature near the proximal end region 362 of the carriage 360 allowing for movement of the carriage 360 in a proximal direction. This depression of the actuator 340 can occurs prior to the second step of retracting the actuator 340 proximally such as described above with FIG. 5E. Alternatively, depression of the actuator 340 withdraws the carriage 360 and the implant cartridge 305 engaged with the carriage 360 relative to the hand piece 310 via the pivoting mechanical linkage 385 as in FIG. 5F. The delivery cannula 320 is affixed to the implant cartridge 305 so that the cannula 320 and the cartridge 305 move together during retraction. The cartridge 305 moves relative to the inner member 335 extending through the lumen 330 of the delivery cannula 320, which remains stationary, causes the implant 10 to be left in position within the desired surgical depth. As mentioned above, the actuator 340 can unlock as a first step and switch into travel mode as a second step. Actuation can additionally include a third step where the cartridge 305 is re-locked following deployment of the implant. FIG. 5G shows the actuator 340 prior to being depressed. The mechanical feature 389 of the safety latch 390 is engaged preventing proximal retraction of the carriage 360. FIG. 5H shows the actuator 340 after being depressed. The mechanical feature 389 at the proximal end region of the elongate lever 387 is lifted away from its engagement with the proximal end region 342 of the carriage 360. The pivoting movement of the mechanical linkage 385 is complete such that the carriage 360 is urged into its proximal position. A button latch 392 that was lifted out of engagement with the carriage 360 in FIG. 5G is now engaged with a detent 394 in the carriage 360 at the end of proximal travel of the carriage 360 because the detent 394 in the carriage 360 aligns with a correspondingly-shaped feature on the button latch 392 allowing the latch 392 to lower into the detent 394 catching it and thereby preventing further movement of the carriage 360 in the proximal direction maintaining it in the fully retracted position. The geometry of the detent 394 and the correspondingly-shaped feature on the button latch 392 are angled so that the resistance can be readily overcome, such as by urging the reset button 369 on the proximal end of the hand piece 310 in the distal direction.

In the implementation described above with respect to FIG. 5E, the actuator 340 is a two-part actuator in which the button is pushed inward to unlock and retracted proximally so that the mechanical linkage urges the carriage 360 proximally to retract the implant cartridge 305. In the implementation described above with respect to FIGS. 5F, 5G-5H, the actuator is a push button with a pivoting mechanical linkage 385 that as the actuator 340 is urged inwardly it pivoted thereby urging the carriage 360 proximally to retract the implant cartridge 305. FIGS. 6A-6C illustrate another implementation of an actuator 340 of a delivery device in which the actuator 340 is pushed distally to retract the implant cartridge 305 proximally. The implant cartridge 305, which is not shown in FIGS. 6A-6C, but can be any of the implant cartridges 305 described herein including those shown in FIGS. 4A, 4I-4J, and 5A can engage with a distal coupler, such as carriage 360 near the distal end region 317 of the housing 314 of the hand piece 310, such as by inserting within an opening 370 of the carriage 360 as described elsewhere herein. A proximal end region 362 of the carriage 360 can be coupled to a proximal end region 342 of the actuator 340 by a mechanical linkage 385 that pulls the carriage 360 proximally as the actuator 340 is urged distally. The configuration of the mechanical linkage 385 can vary including a primary mechanical linkage, a gear slider, pivot only, etc. Generally, regardless the specific mechanical linkage between the actuator 340 and the carriage 360, the linkage 385 is intended to provide a mechanical advantage to take the large movements by a surgeons and provide proper resolution over a shorter stroke of implant deployment into the canal.

FIGS. 6D-6E are partial side views of implementations of actuators 340. FIG. 6D shows the carriage 360 engaged with an implant cartridge 305. The mechanical linkage 385 illustrated is a gear slider. Sliding the actuator 340 distally pulls the carriage 360 proximally. The mechanical linkage 385 can be a rack and pinion mechanism with 2:1 gearing between the rack wheels. FIG. 6E shows the mechanical linkage 385 is a lever mechanism that reverses the motion. The lever can have a 2:1 advantage discussed above. The mechanical linkage 385 can incorporate pin and slot rotation to reduce sliding parts in the actuation of the device.

The distance of retraction provided by any of the actuators 340 described herein can vary. The mechanical linkage 385 for the actuator 340 (including any of the actuators 340 shown in FIGS. 4B, 5B, 5E-5G, 6A-C, 6D-6E) can provide a 1:1 ratio between actuator movement and cartridge retraction to deploy the implant 10 such that the distance the actuator 340 moves (e.g., slides, pivots, etc.) is equal to the distance the cartridge retracts. For example, the motion of the actuator 340 (e.g., 1 mm-10 mm distance) can result in cartridge motion of the same distance to deploy the implant. In some implementations, the distance of retraction provided is about 1 mm-5 mm, preferably about 2 mm-4 mm to ensure full travel of the delivery cannula 320 over the inner member 335 to release the implant 10. Preferably, larger movements of the surgeon to move the actuator 340 provides a shorter stroke of cartridge region for implant deployment in the eye. For example, the ratio of actuator motion to cartridge motion can be a 1:1, 2:1, 3:1, 4:1 ratio and anywhere in between, such that the motion of the actuator 340 is amplified relative to the cartridge motion achieved to provide a shorter stroke for implant deployment.

After deployment of an implant from the implant cartridge 305, the tissue cartridge 305 can be removed from the hand piece 310 and the hand piece 310 used again for deployment of another implant. Any of the actuation mechanisms described above can be reset such as by urging the reset button 369 inward through the housing 314, which urges the carriage 360 distally compressing a spring (not shown) until the safety latch 390 is once again engaged and the mechanical feature 389 engages the feature near the proximal end region 362 of the carriage 360 thereby arming the device for further deployments.

The implant(s) 10 are preferably implanted ab interno through a clear corneal or scleral incision or puncture using the delivery device 300 described above. The distal end region 380 of the delivery cannula 320 is advanced through a clear corneal or scleral incision or puncture to access the anterior chamber. In this regard, the opening can be made in the eye, such as within the limbus of the cornea. In an implementation, the opening is close to the limbus, such as either at the level of the limbus or within 2 mm of the limbus in the clear cornea. Preferably, a separate cutting device can be used to create the opening (i.e., incision or puncture), for example, a knife-tipped device or diamond knife can be used initially to enter the cornea. A second device with a spatula tip can then be advanced over the knife tip wherein the plane of the spatula is positioned to coincide with the dissection plane. The spatula tip device can be the delivery cannula 320.

The opening can have a size that is sufficient to permit passage of the delivery cannula 320, but that is minimally-invasive and preferably self-sealing. In an implementation, the opening is about 1 mm in size. In another implementation, the opening is no greater than about 2.85 mm in size. In another implementation, the opening is no greater than about 2.85 mm and is greater than about 1.5 mm. It has been observed that an opening of up to 2.85 mm is self-sealing.

A viewing lens such as a gonioscopy lens can be positioned adjacent the cornea. The viewing lens enables viewing of internal regions of the eye, such as the scleral spur and scleral junction for implanting within a uveoscleral outflow pathway or the trabecular meshwork for implanting within Schlemm's canal, from a location in front of the eye. The viewing lens may optionally include one or more guide channels sized to receive the cannula 320. An endoscope can also be used during delivery to aid in visualization. Ultrasonic guidance can be used as well using high-resolution bio-microscopy, OCT, and the like. Alternatively, a small endoscope can be inserted through another limbal incision in the eye to image the eye during implantation.

After insertion through the opening, the delivery cannula 320 can be advanced into the anterior chamber along a pathway that enables the implant 10 to be delivered ab interno from the anterior chamber into the target location, such as the uveoscleral pathway (i.e., supraciliary or suprachoroidal space) or the trabecular pathway (i.e., Schlemm's Canal). Each of which will be described in more detail below.

For positioning within the uveoscleral pathway, the delivery cannula 320 positioned for approach is advanced further into the eye such that the distal-most tip of the delivery cannula 320 penetrates the tissue at the angle of the eye, for example, the iris root or a region of the ciliary body or the iris root part of the ciliary body near its tissue border with the scleral spur. The scleral spur is an anatomic landmark on the wall of the angle of the eye. The scleral spur is above the level of the iris but below the level of the trabecular meshwork. In some eyes, the scleral spur can be masked by the lower band of the pigmented trabecular meshwork and be directly behind it. The delivery cannula 320 can travel along a pathway that is toward the angle of the eye and the scleral spur such that the delivery cannula 320 passes near the scleral spur on the way to the supraciliary space but does not necessarily penetrate the scleral spur during delivery. Rather, the delivery cannula 320 can abut the scleral spur and move downward to dissect the tissue boundary between the sclera and the ciliary body, the dissection entry point starting just below the scleral spur near the iris root or the iris root portion of the ciliary body. In another implementation, the delivery pathway of the implant intersects the scleral spur.

The delivery cannula 320 can approach the angle of the eye from the same side of the anterior chamber as the deployment location such that the delivery cannula 320 is not advanced across the iris. Alternately, the delivery cannula 320 can approach the angle of the eye from across the anterior chamber AC such that the delivery cannula 320 is advanced across the iris and/or the anterior chamber toward the opposite angle of the eye. The delivery cannula 320 can approach the angle of the eye along a variety of pathways. The delivery cannula 320 does not necessarily cross over the eye and does not intersect the center axis of the eye. In other words, the corneal incision and the location where the implant 10 is implanted at the angle of the eye can be in the same quadrant when viewed looking toward the eye along the optical axis. Also, the pathway of the implant 10 from the corneal incision to the angle of the eye ought not to pass through the centerline of the eye to avoid interfering with the pupil.

The delivery cannula 320 can be continuously advanced into the eye, for example approximately 6 mm. The dissection plane of the delivery cannula 320 can follow the curve of the inner scleral wall such that the distal end region 380 of the delivery cannula 320, for example after penetrating the iris root or the iris root portion of the ciliary body CB, can bluntly dissect the boundary between tissue layers of the scleral spur and the ciliary body CB such that a distal end region 380 of the delivery cannula 320 extends through the supraciliary space and then, further on, is positioned between the tissue boundaries of the sclera and the choroid forming the suprachoroidal space.

Once properly positioned, the implant 10 can be released from the delivery cannula 320 as described herein. In an implementation, the implant 10 is urged distally through the lumen 330 out the distal opening 325 from the delivery cannula 320. In a preferred implementation, the delivery cannula 320 is retracted over the inner member 335 located proximal to the implant 10 maintaining the implant 10 stationary relative to the hand piece 310 until the implant 10 is exposed outside the distal opening 325 of the delivery cannula 320. In still further implementations, the implant 10 is urged distally simultaneously or prior to as the delivery cannula 320 being retracted proximally.

The distal end region 380 of the cannula 320 can be used to create a cleft between tissues at the location of implantation. The distal end region 380 of the cannula 320 can incorporate one or more visual markers to guide a user regarding depth of penetration between tissue layers. A first marker can be positioned a minimum depth of penetration desired (e.g., about 3 mm from the distal-most tip of the cannula). A second marker can be located a distance further proximal of the first marker for a second maximum depth of penetration desired (e.g., about 6 mm from the distal-most tip). Once the user has created the desired cleft and is ready to deliver the implant 10 from the lumen 330, the inner member 335 remains fixed and the delivery cannula 320 is retracted over it. Retracting the delivery cannula 320 while the inner member 335 remains fixed releases the implant 10 from the lumen 330. The visual markers can be generated using Physical Vapor Deposition methods of coating.

The implant 10 can be deployed from the lumen 330 so that at least a portion of the implant 10 is positioned between tissue layers, such as within a supraciliary space between ciliary tissue and scleral tissue, or within Schlemm's Canal either transmurally or circumferentially, as described in more detail below. The implant 10 can be deployed so that it is positioned within the supraciliary space so that at least a distal region is positioned between ciliary tissue and the sclera, and a proximal end is within the supraciliary space, within the supraciliary cleft, or within the anterior chamber. The proximal end need not project into the anterior chamber when positioned within the supraciliary cleft. Preferably, the proximal end of the implant 10 is positioned so that it remains flush with the cleft positioned between ciliary tissue and scleral tissue and does not extend into the anterior chamber. In another implementation, the implant 10 (or implants) are deployed so that they are positioned within Schlemm's Canal so that at least a distal region of the implant is positioned within the canal and a proximal end region of the implant is positioned within the canal, within the trabecular meshwork, or within the anterior chamber. In some implementations, the implant swells or expands upon deployment and rehydration such that the size of the implant becomes larger than a natural cross-sectional dimension of Schlemm's canal. The implant can enlarge so that it no longer is entirely contained within the canal and may encroach upon the trabecular meshwork and/or the anterior chamber as it reaches its fully deployed shape and configuration.

Once implanted, the implant 10 forms a fluid communication pathway from the anterior chamber to the target outflow pathway (e.g., supraciliary space or suprachoroidal space or Schlemm's Cana, la subconjunctival location of the eye, retinal or subretinal location, intravitreal or posterior chamber, and other ocular locations. In another implementation, the implant 10 is implanted to form a fluid communication pathway between the anterior chamber and the Schlemm's Canal and/or communication pathway between the anterior chamber and a subconjunctival location of the eye. The device described herein can also be used to deliver an implant trans-sclerally as well from an ab interno approach.

The implant 10 forming a fluid communication pathway can be supplemented by an additional implant, such as an implant formed of the same tissue as the first implant or a different tissue as the first implant.

As discussed elsewhere herein, the biologically-derived material can include tissues, such as scleral tissue, corneal tissue, or amniotic membrane tissue. In preferred implementations, the biologically-derived material is for implanting within Schlemm's Canal is scleral tissue so that the tissue is homologous to the outer scleral wall of the canal. The tissue implant 10 positioned within the canal is hydrophilic and has a porosity that can create a wick for improved outflow from the anterior chamber, even when a single implant is positioned within the canal.

The delivery cannula 320, along with being a storage container for the cut and processed implant 10, can be used for positioning the implant in the eye. The implant 10 can be compacted within the delivery cannula so that the size of the implant inside the lumen is smaller than the cut size of the implant, particularly following deployment of a dehydrated implant in the eye. Upon release of the implant into a location in the eye the implant rehydrates and increases in size. The increase in size can be due, in part, to rehydration of the tissue and/or the implant relaxing back into its cut dimension effectively “stents” the space within which it is deployed. The implant can also be sized relative to the dimensions of the delivery cannula so that there is no compacting or compression of the implant upon loading the implant within the lumen of the delivery cannula.

In some implementations, the implant can be configured to change shape following deployment due to hydration of the tissue in the eye. The implant can be cut from a dehydrated bio-tissue, such as dehydrated cornea or dehydrated sclera or dehydrated amniotic membrane. The dimensions of the cut dehydrated tissue can be smaller than corresponding hydrated tissue. A narrower cut can allow for smaller delivery cannula dimensions (e.g., inner diameter as small as 200-300 microns). Smaller cannulas are less traumatic to the eye and result in a smaller cleft size during implantation that upon deployment of the tissue can be propped up by expansion of the implant as it rehydrates in situ. In some implementations, the implant 10 is packaged so as to be bathed within a liquid solution. The implant 10 can be loaded into the delivery cannula 320 using a liquid solution as described above. In other implementations, the implant 10 is dehydrated and is loaded “dry” into the delivery cannula 320. In some implementations, the tissue can be transferred into the cannula manually using tweezers or another loading tool such as a physical pusher and inserted directly into the lumen. The dehydrated implant 10 allows for the delivery cannula dimension to remain small, for example, to maintain a small corneal incision. Once the implant 10 is deployed from the delivery cannula 320 into the eye, exposure to the bodily fluids rehydrates the implant 10 causing the implant 10 to swell and expand. Expansion of the implant 10 during hydration stents open the space within which the implant 10 is positioned to improve outflow of aqueous through the canal.

The inner diameter of the delivery cannula 320 can accommodate an implant 10 having a width of about 400-800 microns. Upon deployment of the implant 10 in the eye, the bio-tissue of the implant 10 can expand upon hydration to a size that can be about 50%-200% greater than the initial dehydrated volume. The implant 10, upon deployment, can increase in size by at least 1.2×, at least 1.3×, at least 1.4×, at least 1.5×, at least 1.6×, at least 1.7×, at least 1.8×, at least 1.9×, at least 2× up to about 3× the original size of the implant and anywhere in between.

More than a single implant 10 can be deployed within the eye to aid in aqueous outflow from the anterior chamber including implants 10 that are configured to enlarge in dimension upon deployment. The implants 10 can be formed of the same type of material or can be different materials. In some implementations, a first implant 10 is formed of scleral tissue and is implanted as a primary tissue implant and a second implant 10 is formed of amniotic membrane tissue and is implanted as a secondary tissue implant that acts as an adjunct treatment adjacent the primary tissue implant such as by releasing one or more healing factors in the implant location of the first implant 10.

Implanting more than one implant within a target site can also create a scaffold or reservoir within the target site. A pair of implants 10 can be positioned within the anterior angle of the eye creating a scaffold or reinforced segment within Schlemm's Canal. The pair of implants 10 can be located side by side with a longitudinal space between them that forms a tented volume or reservoir between them. The longitudinal space forming the reservoir between the two implants 10 can be a permanent or semi-permanent reservoir that increases the outflow of aqueous from the anterior chamber. A first implant can be positioned within a first region of Schlemm's Canal, such as a dilated segment of the canal, and at least a second implant is positioned within a second region of Schlemm's Canal adjacent to the first implant (see FIGS. 2A-2B).

A first implant 10, which can be an elongate segment of scleral tissue or other tissue as described elsewhere herein, can be inserted from the delivery cannula into an aqueous drainage location, such as a region of Schlemm's Canal, so that the distal end of the first implant 10 is in fluid communication with the Schlemm's Canal and the proximal end of the first implant 10 remains at least in part within the anterior chamber, within the trabecular meshwork, flush with the canal, or fully contained within the canal. Optionally, a second implant 10, which can also be an elongate segment of scleral tissue or can be another tissue such as corneal tissue, amniotic membrane, and the like, serving as an adjunct treatment for the primary implant 10, is inserted along a similar trajectory so the distal end of the second implant 10 is placed in fluid communication with the Schlemm's Canal within a distance away from the first implant 10 within the canal. The proximal end of the second implant 10 can be in fluid communication with the anterior chamber, within the trabecular meshwork, flush with the canal, or entirely within the canal. The second implant 10 can be delivered using the same delivery cannula 320 loaded at the site of the procedure or a different delivery cannula 320 preloaded with the second implant 10. The second implant 10 can be spaced a distance away from the first implant 10 around a circumference of the eye. The distance between the first and second implants 10 can vary, but is preferably about 0.5-2.0 mm away to scaffold the tissues extending longitudinally between the two implants 10 and, in the case of adjunct treatment by eluting healing factors derived from the second implant ensures the factors diffuse to the implant site without significant wash-out. As an example, at 0.5 mm, each implant is about 5 degrees on a 12 mm diameter circle or 20 degrees if 2 mm apart.

The tissue implant can be a resorbable tissue and semi-permanent to create the scaffold for a limited period, such as 1 month to 3 months. The implant can be used to create an expandable scaffold within the target site that after about 30 days absorbs leaving a functional and improved outflow (e.g., ciliary cleft or trabecular meshwork/canal) with a more open architecture. The implant can be amniotic membrane used in addition to one or more scleral tissue implants in the treatment location to reduce fibrosis by eluting healing factors derived from the amniotic membrane that diffuse in and around the implantation location inhibiting fibrosis and increasing regeneration and healing. In some cases, a tissue implant described herein can be a permanent implant (or plurality of implants), for example, wherein the implant is not removed after implantation at the target site.

As an example procedure, an eye of a patient with glaucoma can be prepped and draped using various technique. Retrobulbar or sub-tenon block can be delivered under Monitored Anesthesia Care (MAC). A first incision is made in the cornea and viscoelastic injected to deepen the anterior chamber. An intraoperative gonio-lens is applied to the cornea to visualize the angle, which is expanded additionally to visualize under the microscope and gonio-lens the Schlemm's Canal near the iris/ciliary body root insertion. Schlemm's Canal dilation can be performed using micro-interventional instrumentation described herein, for example, using the dilation system 60 including a dilation tool 600 and/or a fluid delivery device 601. One or more dilated segments of Schlemm's Canal can be created prior to insertion of one or more implants 10. A second corneal incision can be made to deliver a bio-tissue implant 10. The implant 10 can further expand and scaffold the segment of the canal. Optionally, a second bio-tissue implant 10 can be delivered, such as to the opposite lateral segment of Schlemm's Canal to scaffold and maintain the dilated segment. As described elsewhere herein, the distance between the implants 10 can vary, but is preferably a distance no more than about 2 mm away so as to maintain the scaffold. Bleeding can be minimized using tamponade. The reinforced dilated segment of Schlemm's Canal between the first and second implants 10 can be visualized and confirmed at the conclusion of the case using gonio-microscopy. Irrigation/aspiration can be performed to evacuate any debris or viscoelastic. Antibiotics and steroids can be delivered at the conclusion of the case. The procedure described above relates to just two implants paired to scaffold one or more segments of Schlemm's Canal although the procedure can include positioning three or four implants to create additional scaffolded segments.

Schlemm's Canal has an inner diameter of about 100 microns. Schlemm's Canal can be dilated prior to positioning an implant 10 in the canal. The dilation can be performed using a minimally-invasive mechanical dilation tool 600. The tool 600 can be inserted ab interno into at least a segment of Schlemm's canal to mechanically dilate and stretch at least a segment of the canal as it is advanced through the canal and prior to positioning the implant(s) 10 within the dilated segment. The trabecular meshwork along the dilated canal segment can remain intact during and after stretching so that the dimension of the canal is enlarged yet still fully enclosed. The mechanical dilation can be performed without the use of fluid-mediated dilation, such as without any viscoelastic delivery to the canal. Viscoelastic can optionally be used to dilate the canal prior to insertion of the implant 10. For example, a cannula can be coupled on a proximal end region to a syringe containing ophthalmic viscoelastic solution. At least a distal opening of the cannula can insert through trabecular meshwork to access the canal for injection of the solution through at least a portion of the canal. The fluid injection can be in additional to or instead of mechanical dilation of the canal prior to implantation. The cannula can be a 30 gauge cannula with a 5 mm beveled tip. Other systems or techniques are considered herein to dilate a segment of Schlemm's canal prior to implant deployment, including, for example, OMNI Surgical System (Sight Sciences, Inc., Menlo Park, CA).

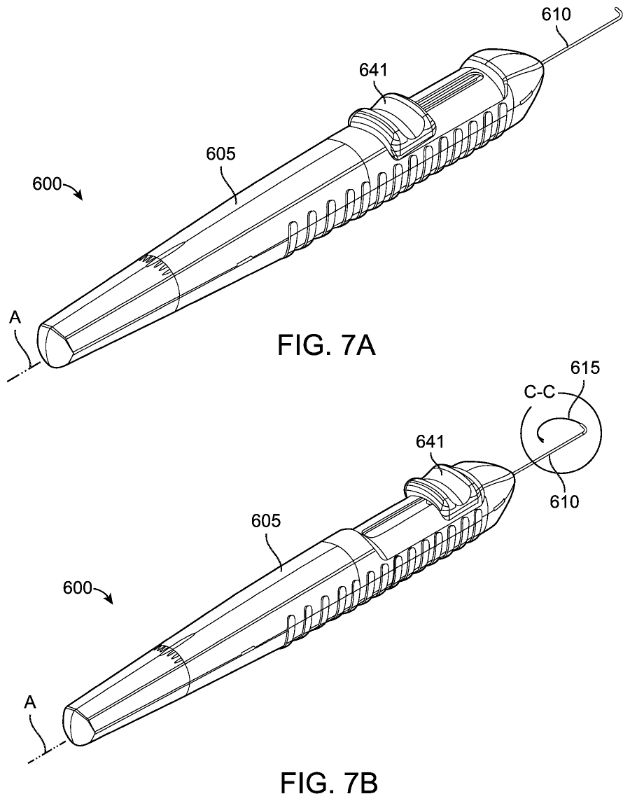

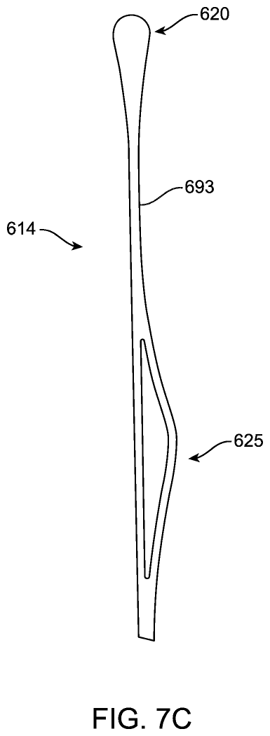

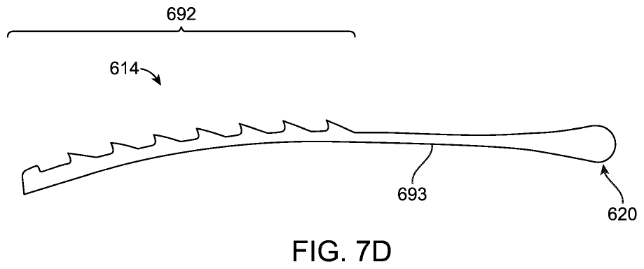

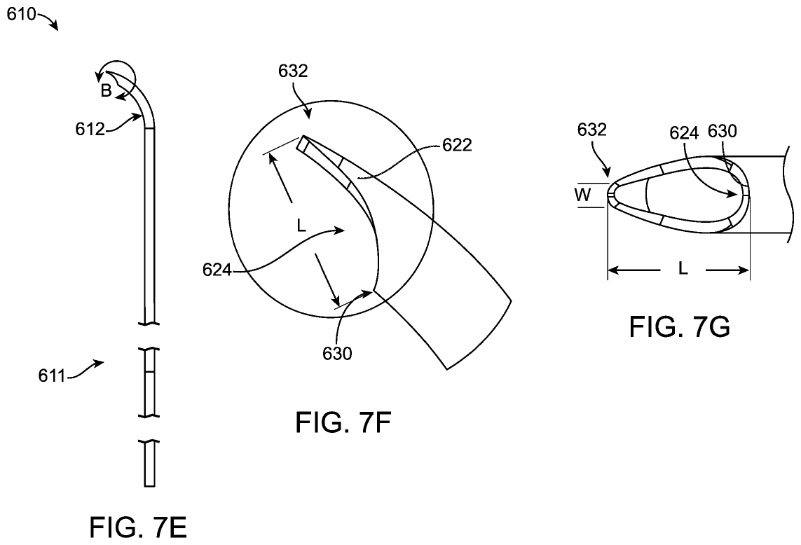

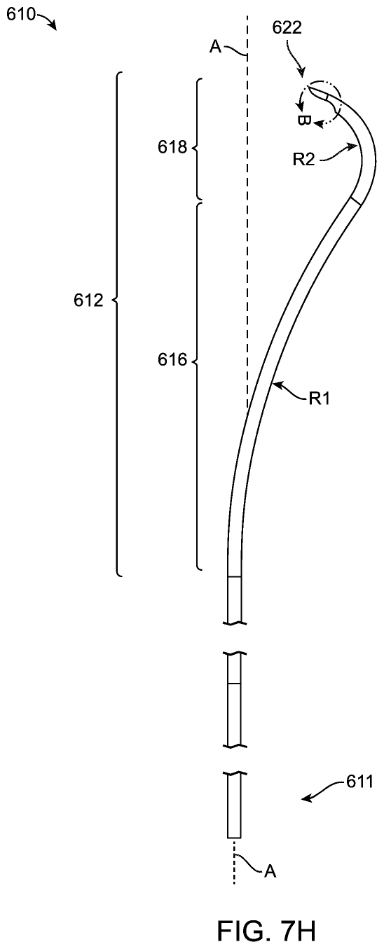

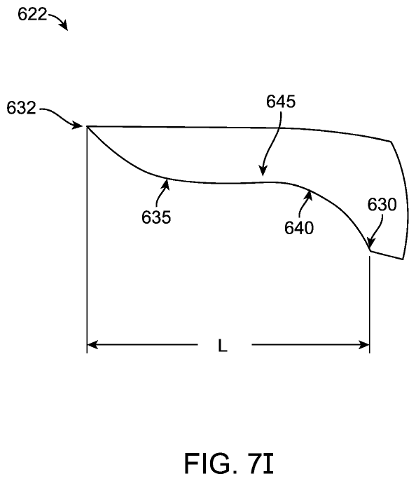

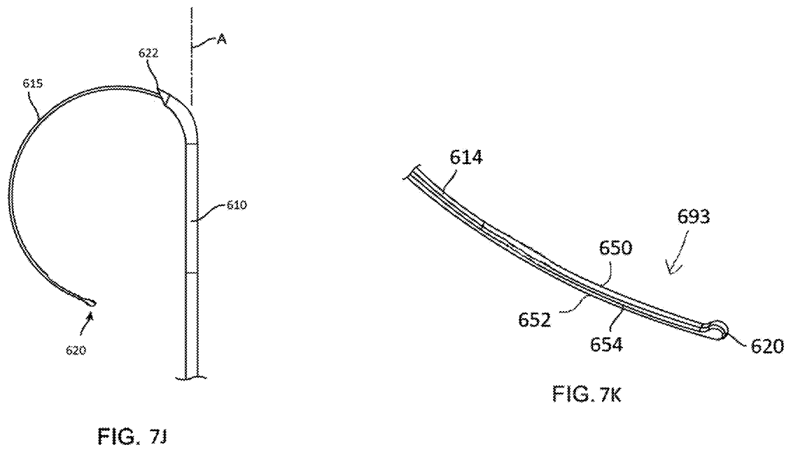

FIGS. 7A-7B illustrate the dilation tool 600. The mechanical dilation tool 600 can incorporate a proximal hand piece 605 having an outer shaft 610 projecting distal to the proximal hand piece 605 and an inner shaft 615 configured to be advanced and retracted through the outer shaft 610 relative to the proximal hand piece 605 using an actuator 641, such as a slider. FIG. 7A shows the inner shaft 615 retracted inside the outer shaft 610. FIG. 7B shows the inner shaft 615 fully extended relative to the outer shaft 610. The outer shaft 610 is generally rigid and designed to maintain its shape during use. The inner shaft 615 is elongate and flexible so that it can be extended out the distal end region of the outer shaft 610 for insertion within Schlemm's Canal. Both the inner shaft 615 and the outer shaft 610 are sized and configured for ab interno insertion into the anterior chamber. In use, the inner shaft 615 can be retracted fully inside the lumen of the outer shaft 610 so that the distal-most end of the outer shaft 610 can be used to penetrate through the corneal puncture to access the anterior chamber and penetrate through the trabecular meshwork to access Schlemm's Canal. The outer shaft 610 can be used to create an opening in the trabecular meshwork to access Schlemm's canal. FIG. 7G illustrates a tip region of the outer shaft 610 that is capable of being used to puncture the trabecular meshwork first prior to advancing the distal-most end 620 of the inner shaft 615 through the canal. The tip of the outer shaft 610, which extends distal to the opening into the lumen of the shaft 610 can be smaller than about 200 microns across. Alternatively, another tool can be used to create the opening.

The inner shaft 615 can be a round wire or flat ribbon without a lumen extending through it (e.g., polyimide or Nitinol sheet cut or stamped to shape). The shaft 615 may be laser-shaped so as to have a non-circular cross-section and a distal end region comprising any of a variety of profiles, which will be described in more detail below. The cross-sectional shape of the shaft 615, taken transverse to a length of the inner shaft, can be square or rectangular. The non-circular cross-section of the shaft 615 can include a radially inward surface 650 connected to a radially outward surface 652 by two lateral sides 654 (see, e.g., FIG. 7K). The distal end can have rounded distal-facing edges formed into a smooth ball tip 620 that is blunt for circumferential gonio-traction. The rounded edges on the distal face of the shaft 615 allow for better sliding relative to the tissue compared to square edges. The inner shaft 615 can be a tubular element having a lumen (e.g., polyimide or Nitinol hypotube).

The inner shaft 615 can have any of a variety of designs as described in U.S. Patent Publication No. 2025/0169989, filed Nov. 22, 2024, which is incorporated herein, that stretch or dilate Schlemm's canal while keeping the trabecular meshwork substantially intact.

The distal portion of the shaft 615 can be shaped into a curve forming a curved distal portion having a central plane (e.g., about 220 degrees-230 degrees). The distal portion of the inner shaft 615 can be straight. Whether curved or straight, the distal portion of the inner shaft 615 can provide tracking around Schlemm's canal with a minimum amount of effort or resistance.

The inner shaft 615 can incorporate markers on at least one surface of the shaft 615. As an example, the markers can be positioned on an inner-facing surface of the shaft 615 and arranged for every clock hour of insertion around the canal. The inner-facing surface of the inner shaft 615 can be radially inward relative to the eye as the inner shaft 615 is advanced through the canal. The surface with the one or more markers is preferably a surface of the inner shaft 615 that is visible to a user as the inner shaft 615 is advanced relative to the outer shaft 610. An outer-facing surface of the inner shaft 615 opposite the inner-facing surface is generally less visible during use due to it facing towards and/or abutting against the outer wall of Schlemm's as the inner shaft 615 is advanced through the canal. The one or more markers can provide visual guidance to indicate the degree of advancement of the inner shaft 615 relative to the outer shaft 610 so as to provide information regarding length of segments of the canal that are dilated by the shaft 615 from the location of insertion of the shaft into Schlemm's Canal. Any of a variety of surface features of the inner shaft 615 can be located along the inner shaft 615 in a metered manner to act as visual markers to indicate depth of penetration relative to Schlemm's Canal and thus the degree of advancement of the inner shaft relative to the hand piece 314 and/or relative to the outer shaft 610, such as out the distal opening of the outer shaft 610. The surface features can be metered markers as discussed above as well as one or more other features including the dilating portion 625, the plurality of teeth 692, and any surface coatings incorporated on the inner shaft 615 that are visible to a user.

At least a portion of the distal end region of the inner shaft 615 can have an outer dimension sized larger than 100 microns to stretch and dilate Schlemm's Canal without tearing the trabecular meshwork thereby leaving the trabecular meshwork of the dilated segment of the canal intact at least upon advancement of the tissue dilator portion of the inner shaft 615 through the canal.

The region of the inner shaft 615 that performs the stretching and dilating of Schlemm's Canal can be the distal-most end 620. Meaning, the distal-most end 620 can form the maximum outer dimension of the inner shaft 615 along its length such that the inner shaft 615 need not incorporate any other dilation portion proximal of its distal-most end 620 (see, e.g., FIGS. 7J and 7K). Any of the dilation tools described herein incorporates an enlarged distal-most end 620 and no other enlarged region along its length proximal of the distal-most end. Preferably, the inner shaft 615 tapers proximally from the enlarged distal-most end 620 to be thinner for at least a length proximal to the distal-most end 620 forming a neck region 693 (see, e.g., FIGS. 7C, 7D, and 7K). The dilation tools incorporating the enlarged distal-most end 620 can optionally incorporate a dilator portion 625 and/or a plurality of teeth 692 located proximal to the neck region 693 (see, e.g., FIGS. 7C and 7D, respectively).

The region of the inner shaft 615 performing the stretching and dilating can have a maximum thickness along a lateral side 654 between an outward-facing surface 652 and an inward-facing surface 650. Use of the terms “outward” and “inward” are relevant to a direction the surfaces may face during use within the eye. As described elsewhere herein, the shaft 615 is designed to track along at least a segment of Schlemm's Canal. An outward-facing surface of the shaft 615 is one that, in use, is designed to face towards an outer wall of Schlemm's and an inward-facing surface of the shaft 615 faces away from the outer wall of Schlemm's, such as towards the trabecular meshwork of the segment as the inner shaft 615 is advanced through the canal. The maximum thickness of the stretching and dilating region of the inner shaft 615 (e.g., distal-most end 620 and/or the dilating portion 625) along the lateral side 654 from the first surface (e.g., outward-facing 652) to the second surface (e.g., inward-facing 650) can be greater than 100 microns up to about 500 microns, preferably 150-300 microns, most preferably about 200-250 microns, or about 240 microns thick. The inner shaft 615 having a portion that is in this range provides a good balance between being able to enter Schlemm's Canal through the trabecular meshwork, track along at least a portion of Schlemm's Canal, and dilate the canal while maintaining the trabecular meshwork of the dilated canal segment intact.

The distal end region of the inner shaft 615 is inserted into the canal through the trabecular meshwork and actuated to extend relative to the hand piece 605. As the inner shaft 615 extends it advances through Schlemm's canal along at least a segment. The outer dimension of at least a portion of the inner shaft 615 near the distal end region 614 can be at least 150 microns to dilate the segment of the canal it extends through and preferably less than about 400 microns to avoid tearing through the trabecular meshwork as it moves. The distal-most end 620 of the inner shaft 615 can have a rounded shape that is atraumatic and designed to atraumatically slide along the outer wall of the canal as the inner shaft 615 is advanced. FIG. 7C is a detailed view of an implementation of the distal end region of the inner shaft 615 and FIG. 7D is a detailed view of another implementation of the distal end region of the inner shaft 615. The implementation in FIG. 7C is shown having a dilation portion 625 located proximal of the distal-most end 620 having a triangular shape. The implementation in FIG. 7D is shown having a plurality of teeth 692 located proximal of the distal-most end 620 in lieu of the dilation portion 625 as in FIG. 7C. The plurality of teeth 692 can be located proximal of the distal-most end 620, for example, projecting from a region of an outward-facing surface of the inner shaft 615. It should be appreciated that the dilation portion 625 and the plurality of teeth 692 or any other features present on the outer and/or inner walls of the inner shaft 615 are optional. As such, the enlarged dimension of the distal-most end 620 alone is sufficient for the implementations of FIGS. 7C and 7D to dilate and stretch Schlemm's Canal as described herein.

Again with respect to FIG. 7C, the dilation portion 625 of the tool 600 proximal of the distal-most end 620, which is an optional feature of the tool 600, can have an enlarged dimension that also stretches the canal without tearing completely through the trabecular meshwork as it is advanced. FIG. 7D is a detailed view of another implementation of the distal end region of the inner shaft 615 without the triangular-shaped dilation portion 625 as shown in FIG. 7C, but instead includes the plurality of teeth. The plurality of teeth 692 can have a thickness that is greater than about 100 microns up to about 200 microns, or about 140 microns-150 microns, or about 145 microns where the thickness is along a lateral side 654 of the inner shaft 615 from the outward-facing surface 652 to the inward-facing surface 650. The distal-most end 620 of the inner shaft 615 has an enlarged dimension that, alone or together with the plurality of teeth, stretches the canal without tearing completely through the trabecular meshwork as it is advanced. FIG. 7C is a side view of a distal end region of an inner shaft 615 such that the outward-facing surface 652 is not visible, but would face towards a right side of FIG. 7C. Similarly, the inward-facing surface 650 is not visible, but would face towards a left side of FIG. 7C. FIG. 7D is also a side view of the distal end region of an inner shaft 615 such that the lateral side is visible and the outward-facing surface 652 and inward-facing surface 650 are not visible. The outward-facing surface 652 would face towards a top of FIG. 7D and the inward-facing surface would face towards a bottom of FIG. 7D. FIG. 7K is a perspective view of a distal end region of an inner shaft 615 showing the outward-facing surface 652 and the inward-facing surface 650 as well as one of the lateral sides 654.

The distal-most end 620 of the inner shaft 615 has a rounded shape that is atraumatic and designed to slide along the outer wall of the canal as the inner shaft 615 is advanced. The size and shape of the distal-most end 620 are configured for smooth canal non-cutting and non-snagging goniotraction. The shape of the distal-most end 620 can have a width that is at least about 0.12 mm, at least about 0.15 mm, at least about 0.18 mm, at least about 0.20 mm, preferably at least about 0.22 mm, at least about 0.24 mm, up to about 0.30 mm. The width of the rounded bulbous shape of the tip may be described herein as a radius of curvature or a radius of the bulb. Where the shape of the distal-most end 620 is described as bulbous or ball shaped, it should be appreciated that the ball shape may only be within a certain 2-dimensional plane where the shape has a diameter across that is the maximum thickness described above.

The inner shaft 615, whether curved or straight at rest, can take on a curvature of the eye anatomy during use. As such, the inner shaft 615 as it is advanced from the outer shaft 610 as a surface arranged to face anatomy that is positioned radially inward relative to the long axis of the shaft 615 and a surface arranged to face anatomy that is positioned radially outward relative to the long axis of the shaft 615. Use of the terms “radially inward” and “radially outward” are intended to provide relative orientation. The surface of the inner shaft 615 that is radially outward and arranged to face the outer wall of Schlemm's canal can be smooth, such as shown in FIG. 7C, or can optionally incorporate one or more surface features, as shown in FIG. 7D. The one or more surface features can include tines or teeth 692 projecting outward. The teeth 692 can aid in directing the distal-most end 620 of the shaft 615 through the canal at an angle (along with the cannula distal end region) that is better for avoiding engagement with the trabecular meshwork. In some implementations, the teeth 692 are arranged a distance proximal of the distal-most end 620 that is at least about 1 mm, at least about 1.2 mm, 1.3 mm, 1.4 mm, 1.5 mm, 1.6 mm, 1.7 mm, 1.8 mm, 1.9 mm, up to about 2.0 mm proximal of the distal-most end 620. The shaft 615 can incorporate 1, 2, 3, 4, 5, 6, 7, 8, 9, up to about 10 teeth 692. Each can be about 0.15-0.20 mm long and about 0.05-0.10 deep.

In use, the teeth 692 can be directed outward toward an outer wall of Schlemm's canal. The leading surface of each of the teeth 692 (i.e., the distal-facing surface) can be smooth so as to slide along the outer wall of Schlemm's Canal during advancement of the shaft 615 without causing tissue disruption or damage. The trailing surface of each of the teeth 692 (i.e., the proximal-facing surface) can be sharp or serrated or otherwise roughened to catch, cut and/or debride the outer wall of Schlemm's as the shaft 615 is retracted so as to cause tissue disruption. This arrangement of the teeth 692 forms a one-way tissue disruptor that only alters the tissue of the outer wall during retraction of the shaft 615. The teeth 692 or other optional surface features on the outer surface of the shaft 615 can be shaped and arranged to debride endothelial cells that collect on the outer wall of Schlemm's that can cause issues with clogging the collector channels.

The thickness of the shaft 615 along a lateral side between the radially inward surface and the radially outward surface can vary. FIGS. 7C and 7D illustrate this change in thickness from distal-most end 620 moving proximally. The maximum thickness of the distal-most end 620 is greater than the maximum thickness of a neck region 693 proximal of the distal-most end 620. The dilation portion 625 as in FIG. 7C and the teeth 692 as in FIG. 7D have a maximum thickness that is also greater than the maximum thickness of the neck region 693. The thickness of the shaft along a lateral side between the radially-inward surface and the radially-outward surface within the neck region 693 can be about 50-100 microns, or about 55-95 microns, or about 60-80 microns. The maximum thickness of the distal-most end 620 can be about 100-500 microns, preferably about 150-300 microns, most preferably about 200-250 microns, or about 240 microns to balance entering, tracking, and dilating Schlemm's Canal. The maximum thickness of the dilation portion 625, if present, can have a similar range in thickness as the distal-most end 620. The dilation portion 625 is optional and can be replaced by a plurality of teeth 692. Thus, where the dilation portion 625 is described as being present on the shaft, another type of disruptor can be present in this region proximal of the neck, such as the plurality of teeth. The distal-most end 620 can have a greater maximum thickness (e.g., about 240 microns) than the maximum thickness of the plurality of teeth (e.g., 145 microns). The shape and thickness of the distal-most end 620 relative to the shape and thickness along other parts of the shaft 615 proximal to the distal-most end 620 provides a self-guiding type of trackability along the canal. The shaft 615 can have a flexible column strength for self-guided stretching of the trabecular meshwork and dilation of the canal.

The inner shaft 615 may be formed of metal or plastic materials that have sufficient flexibility at the desired dimensions of the inner shaft 615 to follow the curvature of the eye during advancement from the shaft 610 and are relatively firm and structurally suitable for exerting a force on the tissues forming the canal. Preferably, the material of the inner shaft 615 is Nitinol, but can also be formed of other metals or metal alloys. Plastics such as polyimide are suitable as well.

One or more regions of the inner shaft 615 and/or outer shaft 610 can have a surface modification that creates micro-abrasive surface for scraping, cleaning, debriding, abrading, shaving, perforating, and/or thinning the canal wall, including disrupting the endothelial cell lining of the canal. This can improve access to the collector channels and episcleral plexus from within the canal. The selected surfaces of the inner shaft 615 that are modified can include at least a distal end region of the inner shaft 615 or a surface arranged to engage an outer wall of Schlemm's Canal as the shaft is tracked through the canal. The surface modification can be created by coating the one or more regions of the inner shaft, such as by Physical Vapor Deposition, electroplating, diamond coatings, or other method. The coatings can additionally be used to provide visual indication of depth of penetration of the inner shaft 615 as described elsewhere herein.

FIGS. 7E and 7H are side views of implementations of an outer shaft 610 for the dilation tool 600 of FIG. 7A. The outer shaft 610 can be coupled to and extending distal from the proximal hand piece 605. The outer shaft 610 is a tubular element having an inner lumen. The tube can be a stainless steel hypotube that is about 500 microns in outer diameter or about 0.020″-0.028″. The outer shaft 610 preferably has an outer dimension sized to insert through a clear corneal incision less than about 2.75 mm, or less than about 2.5 mm, about 1 mm. In an implementation, the outer shaft 610 can be a 25 gauge extra thin wall hypotube.

The outer shaft 610 can be substantially straight and extend along the longitudinal axis A of the proximal hand piece 605 along at least a part of its proximal length 611. Preferably, the outer shaft 610 incorporates a curve away from the longitudinal axis A along at least a portion of its distal length 612. The curved distal length 612 of the outer shaft 610 can be about 15-20 mm. The straight proximal length 611 can be about 50-55 mm for a total length of the outer shaft 610 that is about 65-75 mm. The straight proximal length 611 of the outer shaft 610 allows for the curved distal length 612 to access the target site within the anterior angle while the substantially straight portion is maintained at the corneal incision site. The portion of the outer shaft 610 extending through the corneal incision site during use is substantially straight allowing the user to rotate the outer shaft during use without inadvertently torqueing the incisional area.

Optionally as shown in FIG. 7H, the curved distal length 612 can incorporate a dual curve geometry. The dual curve geometry of the outer shaft 610 helps to alleviate wrist pressure and ensure the inner shaft 615 takes a more tangential path out of the distal opening into Schlemm's canal to function more effectively. The dual curve also helps to provide the user with a surface to press into the anterior angle against the inner wall of Schlemm's canal. The proximal length 611 extends away from the hand piece 605 along the longitudinal axis A. An optional first curved region 616 of the curved distal length 612 can curve in a first direction at a first radius of curvature R1. The radius of curvature R1 of the first curved region 616 can be about 20-25 mm. A second curved region 618 of the curved distal length 612 can curve in a second direction at a second radius of curvature R2. The second radius of curvature R2 of the second curved region 618 can be smaller than the first, such as about 2-3 mm. The first curved region 616 can extend along a length of about 10-15 mm. The second curved region 618 can extend along a length of about 3-5 mm. The curves 616, 618 of the outer shaft 610 can work together to bring the distal tip 622 of the outer shaft 610 into a position best suited to enter Schlemm's Canal for advancement of the inner shaft 615 from the outer shaft 610 for dilation of a canal segment. The combined curves can bring the tip 622 of the outer shaft 610 so it is nearly tangential with the curvature of the canal. The first curved region 616 also helps the surgeon's wrist position to be more natural and comfortable without feeling cramped during use.

The result of the dual curves is that the distal opening is off-set a distance from the longitudinal axis A of the proximal length 611 and directed at an angle relative to the axis A. The first curved region 616 curves in a first direction relative to the longitudinal axis A of the proximal length 611 so a radially outer surface of the second curved region 618 is off-set from the longitudinal axis A by about 5 mm-6 mm. The second curved region 618 curves in a second direction relative to the longitudinal axis A of the proximal length 611 so the distal tip 622 of the outer shaft 610 is off-set from the longitudinal axis A by about 2 mm-3 mm.

Whether or not the proximal region of the shaft incorporates a single curve as in FIG. 7E or a dual curve as in FIG. 7H, the curved distal length 612 can form an angle relative to the longitudinal axis A of the straight proximal length 611. The curved distal length 612 can form an angle that is between greater than 90 degrees, preferably about 100 degrees up to about 120 degrees, the angle being between the plane of the distal-most tip 622 of the outer shaft 610 to a plane of the outer surface of the outer shaft 610 proximal to the curved distal end region 612. Angles that are too obtuse, such as greater than about 120 degrees may cause the inner shaft 615 to extend too deeply (steeply) into Schlemm's Canal. This can cause the dilation portion 625 of the inner shaft 615 (or the plurality of teeth 692 if the dilation portion 625 is not present) being inadvertently buried inside of the canal so that it is less capable or incapable of dilating the canal. The angle of the curved distal length 612 can impact the angle of extension of the inner shaft 615 upon extension from the distal opening 624 of the outer shaft 610. An inner shaft 615 extending from an outer shaft 610 having an angle that is about 85 degrees up to about 120 degrees, more preferably about 90 degrees up to about 110 degrees extends at an angle of extension relative to an axis perpendicular to the longitudinal axis A that is less than about 45 degrees, or about 10-40 degrees, preferably about 20-30 degrees and provides the proper depth of penetration for the dilator portion 625 to insert through the canal and dilate the trabecular meshwork. In contrast, the inner shaft 615 extending from an outer shaft 610 having a more obtuse angle that is greater than 120 degrees extends at an angle relative to an axis perpendicular to the longitudinal axis A that is about 45 degrees or greater and provides a depth of penetration of the dilation portion 625 that is more prone to become buried within the canal. Thus, the dilation devices described herein preferably incorporate a curved distal length 612 of the outer shaft 610 that forms an angle relative to the longitudinal axis A of the proximal region of the outer shaft 610 that is between 90 and 120 degrees, preferably greater than 90 degrees and less than 120 degrees, or about 95 degrees, about 100 degrees, about 105 degrees, about 110 degrees, about 115 degrees up to 120 degrees and anywhere in between.

The curved distal length 612 of the outer shaft 610 facilitates insertion of the inner shaft 615 into the canal to direct the dilation portion 625 of the inner shaft 615 in the desired direction along the anterior angle. The angle between the plane of the distal-most tip 622 of the outer shaft 610 to the plane of the outer surface of the outer shaft 610 proximal to the curved distal end region 612 can be at least about 90 degrees, at least about 95 degrees, at least about 100 degrees, at least about 105 degrees, at least about 110 degrees, at least about 115 degrees up to and less than about 120 mm degrees. A more obtuse bend of about 120 degrees or more can result in the inner shaft 615 extending from the outer shaft 610 at an angle that is too obtuse resulting in penetration into the canal that is too deep or too steep such that the dilation portion 625 of the inner shaft 615 gets inadvertently buried inside the canal and is unable to properly dilate the trabecular meshwork. The angle of the curved distal length 612 of the outer shaft 610 may vary depending on what features are incorporated on the distal end region of the inner shaft 615. For example, one or more disruptors on a surface intended to engage with the outer wall of Schlemm's (see teeth 692 in FIG. 7D) if used with an outer shaft 610 having a 90 degree angle at the curved distal length 612 can cause the distal-most end 620 to miss engaging with the trabecular meshwork. A distal curve that is about 100 degree to about 120 degrees enables the ball of the distal most end 620 to better engage with the trabecular meshwork during advancement out the distal opening of the outer shaft 610 even if there are teeth 692 on the outer surface of the shaft 615.

FIG. 7F is a detail view of the outer shaft 610 of FIG. 7E taken at circle B-B and FIG. 7I is a detail view of the outer shaft 610 of FIG. 7H taken at circle B-B showing the distal tip 622 of the outer shaft 610 (which may also be referred to herein as a cannula). In each of FIGS. 7F and 7I, the distal tip 622 is elongated surrounding a distal opening 624 from the lumen of the outer shaft 610 and has a heel 630, a distal point 632, and a length L between the heel 630 and the distal point 632 (see FIG. 7G). The tip 622 has a shape of a spatula sharpened to penetrate or pierce a trabecular meshwork.

The edges forming the tip 622 can be straight, but preferably curve (see FIGS. 7F and 7I). FIG. 7F shows a side view of the edges forming a tip 622 having a curve having a concave shape between the distal point 632 to the heel 630. The radius of curvature of the curve can change along the tip 622. For example, the radius of curvature nearest the distal point 632 can be about 0.08, about 0.50 nearest the heel 630, and about 0.75 around a mid-way point between the distal point 632 and the heel 630. In some implementations, as shown in the side view of FIG. 7I, the edges forming the tip 622 can have a first region 635 forming a first curve having a convex shape and a second region 640 forming a second curve having a concave shape and an inflection 645 between the convex shape of the curve of the first region 635 and the concave shape of the curve of the second region 640. The curve of the first region 635 can extend from the distal point 632 to the inflection 645 between the first and second regions. The curve of the second region 640 can extend from the inflection 645 to the heel 630. The curve of the first region 635 can have a different radius of curvature than the curve of the second region 640. For example, the curve of the first region 635 can have a radius of curvature that is larger than the curve of the second region 640. For example, the radius of curvature of the first region 635 can be about 0.6-0.8 mm and the radius of curvature of the second region 640 can be about 0.4-0.5 mm.

An angle of the tip 622 can be about 35-45 degrees. The length L of the tip 622 from the point 632 to the heel 630 can be about 0.5-2 mm, preferably about 1 mm. The thickness of the tip 622 at the point 632 (the thickness being from an outer surface of the shaft 610 to an inner surface of the shaft 610 at the point 632) can be about equal to the thickness of the wall of the outer shaft 610 (e.g., 0.08 mm). The thickness of the tip 622 increases moving away from the point 632 as the edges of the tip 622 moving towards the heel 630 transition from a partially tubular to a fully tubular shaft. For example, a thickness of the tip 622 about 0.60 mm away from the distal point 632 (e.g., near the inflection 645 of FIG. 7I) can be about 0.25 mm from the outer surface of the outer shaft to an upper surface of the shaft formed by the edges of the spline tip.

The distal point 632 can be sharp to ensure the tip 622 can press into the trabecular meshwork. The width W of the distal point 632 (i.e., from edge to edge) can be about 0.05 mm-0.25 mm or about 0.10 mm-0.20 mm, preferably less than about 0.20 mm down to about 0.15 mm. The outer diameter of the shaft 610 can be about 0.40-0.60 mm. The inner diameter of the shaft 610 can be about greater than a maximum outer dimension of the inner shaft 615 such that all portions of the shaft 615 can fit fully inside the outer shaft 610. The inner diameter of the shaft 610 can be about 0.30-0.40 mm depending on the dimensions of the inner shaft 615 it is being used with.

At least one implant 10 is positioned within the dilated segment of Schlemm's Canal. As discussed elsewhere herein, the biologically-derived material can include tissues, such as sclera, cornea, or amniotic membrane. The tissue is cut into an implant sized and shaped for delivery using a delivery cannula 320 sized, preferably, for ab interno implantation into an eye for trabecular outflow of aqueous, such as the delivery cannula 320 shown in FIGS. 4A-4H). The scleral tissue can be delivered as a small pellet and prepared so it expands upon deployment within the canal due to rehydration of the dehydrated tissue thereby stenting the canal open. Multiple implants can be positioned within the canal forming a large drainage segment of two or three or more biostents implanted around the canal.

The implant 10 can be positioned using the same mechanical dilation tool 600 described above for dilating the canal or can be positioned using a separate delivery tool, such as the delivery tools described elsewhere herein. As an example, at least a portion of the distal end region 380 of the delivery cannula 320 of the delivery device 300 of FIGS. 4A-4C can be inserted through the trabecular meshwork, e.g., through the opening formed during the dilation step, into a dilated segment of Schlemm's canal. The distance the distal end region 380 is inserted into the dilated segment of the canal can vary. For example, the distal end region 380 having a curve can be positioned a distance into the canal segment until the distal opening 325 is fully surrounded by tissue on an inner wall of the canal (i.e., trabecular meshwork) and on an outer wall of the canal (i.e., sclera) prior to deployment (see FIG. 2C). The curved distal end region 380 of the delivery cannula 320 can be inserted into at least a dilated segment of Schlemm's Canal. Alternatively, the curved distal end region 380 can be inserted into a segment of Schlemm's Canal adjacent the dilated segment. The distal opening 325 can be directed away from the outer wall and toward the lumen of the canal so that as the implant 10 is deployed from the cannula 320 the implant 10 is urged a distance circumferentially along the canal so that at least a distal end region of the implant 10 engages the outer wall of the canal and the inner wall of the canal (i.e., the trabecular meshwork) a distance away from where the delivery cannula 320 penetrated the trabecular meshwork. In other implementations, the distal end region 380 of the delivery cannula 320 is straight. When a straight cannula 320 is used to enter the canal, the implant 10 is directed transmurally toward the outer wall of the canal so that a distal end of the implant 10 engages the outer wall of the canal and the proximal end of the implant 10 engages the disrupted portion of the inner wall of the canal where the delivery cannula 320 penetrated the trabecular meshwork (see FIG. 2D).

The implant 10 is positionable within a segment of Schlemm's Canal that is a dilated segment or within a segment that has not been previously dilated. For example, the implant 10 can be implanted within Schlemm's Canal using a delivery cannula 320 as described above without any dilation of the canal. Upon positioning within the eye, the implant 10 becomes rehydrated and begins expanding to a larger size that dilates the segment of the canal within which the implant 10 is deployed. The implant 10 can be a minimally-modified scleral allograft tissue implant that expands more than about 5% in any direction upon hydration to dilate Schlemm's Canal forming a dilated canal segment as it hydrates to increase aqueous outflow from the anterior chamber. The expansion of the tissue implant 10 can cause at least some portion of the implant 10 to penetrate the trabecular meshwork so that the implant 10 is no longer fully contained by the canal, but rather is expanding outside the canal into the trabecular meshwork and in some cases fully through the trabecular meshwork into the anterior chamber. The expansion of the implant 10 into the trabecular meshwork can dilate, stretch, or otherwise apply forces against the trabecular meshwork thereby augmenting outflow of aqueous therethrough due to the newly expanded space provided by the implant 10 in its enlarged state. The expansion of the implant 10 outside the canal into the trabecular meshwork can vary. In some implementations, the implant 10 expands in volume by at least 1.2× its original size up to about 3× its original size and anywhere in between. The implant 10 may expand in at least one dimension by at least about 10 microns up to about 100 microns into and/or through the trabecular meshwork. The starting dehydrated width of the implant 10 may be about 100 nm to about 100 microns and the final rehydrated width may be about 200 microns to about 1000 microns or more so that the implant 10 penetrates the trabecular meshwork along at least a portion of its length enhancing outflow of aqueous through the trabecular meshwork and through Schlemm's canal. It should also be appreciated that at least a portion of the trabecular meshwork may be punctured, penetrated, or removed so that the canal is “unroofed” or no longer a canal along at least a portion of its circumference. The implant 10 positioned within at least a portion of the unroofed segment of the canal and at least a portion of the dilated segment of the canal (dilation provided by the rehydrated implant and/or by a separate mechanical dilation step) can improve aqueous outflow.

The implant 10 can be deployed from the delivery cannula 320 as described elsewhere herein. For example, the implant 10 within the lumen 330 of the delivery cannula 320 can be left within the canal upon retraction of the delivery cannula 320 over the inner member 335 acting as a stopper so that the implant 10 spans the canal between the trabecular meshwork and the outer wall. Alternatively, the implant 10 can be urged out of the lumen 330 of the delivery cannula 320 using a pusher or using a combination of pushing the implant out of the lumen as the delivery cannula is retracted.

In implementations, more than one implant 10 is deployed within Schlemm's canal. A first implant 10 is positioned at a first location within Schlemm's Canal and a second implant 10 is positioned at a second location within Schlemm's Canal a distance away from the first implant 10. The distance can vary, but it is generally greater than the distance along the canal that one implant 10 extends so that a space is present within the canal between the first and second implants. For example, if the implant is 2 mm long and implanted so that the distal end is positioned away from the point of penetration through the trabecular meshwork and the proximal end is located nearest the point of penetration, the next implant is inserted through the trabecular meshwork greater than 2 mm away from the first point of penetration beyond the distal end of the implant. The space between the distal end of the first implant and the proximal end of the second implant can be at least about 0.25 mm, 0.5 mm, 1 mm, 1.5 mm, 2 mm, 2.5 mm, 3 mm, or from about 0.25 mm to about 3 mm. The first and second locations can bound the segment of the canal that was mechanically dilated and create a scaffolded region within the canal that is expanded and dilated for improving aqueous outflow and drainage through the canal. The segment of the canal that is scaffolded has an outer wall formed by the sclera and an inner wall formed by the trabecular meshwork. The trabecular meshwork spanning the scaffolded segment between the first and second implants can be fully intact without any penetrations, cuts, punctures, openings, or other disruptions. The trabecular meshwork spanning the scaffolded segment between the first and second implants can be disrupted, such as with one or more penetrations, cuts, punctures, openings, or other disruptions. At least a portion of the trabecular meshwork spanning the first and second locations between the implants 10 positioned within the canal can be removed up to the entirety of the trabecular meshwork removed. The implants upon rehydration expand in size thereby aiding in fixating the implants in position even where the trabecular meshwork adjacent the implants is removed or disrupted. The implants create pillars of support on either end of the area of trabecular meshwork disruption and bound an enlarged, dilated area with improved outflow through the canal. Each implant on either end of the dilated area extends across the canal so that one surface of the implant contacts the outer wall of Schlemm's canal and one surface of the implant contacts the inner wall of Schlemm's canal. Each implant can be positioned transmurally across the canal, but need not fill the canal so that each implant supports and scaffolds without blocking flow through the canal. More than two implants, for example, three or four or more implants, can be positioned around a circumference of Schlemm's canal so that the implants together create a chain of scaffolded regions within the canal. Each implant can be positioned relative to the neighboring implant within a minimum distance that provides support of the dilated segments of Schlemm's canal.

The implant 10, whether a single implant 10 positioned within Schlemm's canal or more than one implant 10 is positioned within the canal, are preferably dehydrated as described herein. The dehydrated implants 10 can be small in size during delivery through a minimally-invasive opening and rehydrate and expand once inserted into the canal and exposed to aqueous fluid of the eye. The implants 10 upon expansion have improved fixation within the target location. In some implementations, the implant 10 is substantially elongate having a width of about 0.1 mm-0.3 mm, a thickness of about 0.2 mm-0.3 mm, and a length of about 2-4 mm. In other implementations, the implant 10 is a substantially circular disc having a diameter of about 0.1 mm-0.2 mm and a thickness of about 0.15 mm-0.3 mm. Upon rehydration, the implant 10 increases by at least 2× volume as described elsewhere herein. Smaller implants 10 may be delivered using smaller cannula 320. For example, the cannula 320 diameter can be about 200-250 microns for smaller-sized implant pellets.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value. One inch or 1″ corresponds to 2.54 cm (SI-units).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

### P EMBODIMENTS

P EMBODIMENT 1. A method comprising creating a minimally-modified scleral allograft tissue implant that expands more than 5% in any direction upon hydration to increase aqueous outflow from an anterior chamber of an eye.

P EMBODIMENT 2. The method of P EMBODIMENT 1, further comprising dilating Schlemm's Canal forming a dilated canal segment.

P EMBODIMENT 3. The method of P EMBODIMENT 2, wherein dilating Schlemm's Canal comprises using a dilation tool comprising a distal end region having a dimension sized to stretch Schlemm's Canal without tearing a trabecular meshwork as the dilation tool is advanced along Schlemm's Canal.

P EMBODIMENT 4. The method of P EMBODIMENT 2, wherein dilating Schlemm's Canal comprises positioning the minimally-modified scleral allograft tissue implant within Schlemm's Canal in a dehydrated state and forming the dilated canal segment as the implant hydrates.

P EMBODIMENT 5. A device for preparing Schlemm's Canal for receiving a tissue implant, the device comprising: a distal portion sized and configured for ab interno insertion into an anterior chamber of an eye, the distal portion comprising an elongate, flexible shaft comprising a tissue dilator proximal of a distal-most end of the shaft, wherein, during use, the shaft is configured to be advanced along a circumferential contour of Schlemm's Canal, wherein the tissue dilator has a dimension sized to stretch Schlemm's Canal without tearing a trabecular meshwork as the tissue dilator is advanced along Schlemm's Canal forming a dilated canal segment.

P EMBODIMENT 6. The device of claim P EMBODIMENT 5, wherein the distal portion is configured to stretch Schlemm's Canal and maintain trabecular meshwork tissue substantially intact as the flexible shaft is advanced along a circumferential contour of Schlemm's Canal and/or as the shaft is withdrawn along the circumferential contour of Schlemm's Canal.

P EMBODIMENT 7. A system comprising the device of P EMBODIMENT 5 or P EMBODIMENT 6, further comprising the tissue implant configured to be positioned within at least a region of the dilated canal segment for aqueous outflow.

P EMBODIMENT 8. The system of P EMBODIMENT 7, wherein the implant is minimally-modified biological tissue.

P EMBODIMENT 9. The system of P EMBODIMENT 8, wherein the minimally-modified biological tissue is scleral, corneal, or amniotic membrane tissue.

P EMBODIMENT 10. The system of P EMBODIMENT 8, wherein the minimally-modified biological tissue is dehydrated prior to implantation and upon implantation expands more than 5% in any direction upon hydration.

P EMBODIMENT 11. The system of any one of P EMBODIMENTS 7-10, wherein the tissue implant in a dehydrated state is about 50 microns-300 microns.

P EMBODIMENT 12. The system of P EMBODIMENT 11, wherein the tissue implant in a rehydrated state increases in size by about 5%-50%.

P EMBODIMENT 13. The system of any one of P EMBODIMENTS 5 to 12, further comprising an implant delivery system comprising a delivery cannula, the tissue implant being contained within a lumen of the delivery cannula.

P EMBODIMENT 14. A method of increasing aqueous outflow through Schlemm's Canal, the method comprising dilating at least a segment of Schlemm's Canal to stretch at least a portion of a trabecular meshwork.

P EMBODIMENT 15. The method of P EMBODIMENT 14, further comprising inserting at least a first tissue implant within the segment of Schlemm's Canal, the first tissue implant being minimally-modified scleral allograft.

P EMBODIMENT 16. The method of P EMBODIMENT 15, wherein the first tissue implant expands upon deployment within the segment by more than 5% in any direction upon hydration to increase aqueous outflow from the anterior chamber through Schlemm's Canal.

P EMBODIMENT 17. The method of P EMBODIMENT 15 or P EMBODIMENT 16, further comprising inserting a second tissue implant within the segment of Schlemm's Canal, the second tissue implant being minimally-modified scleral allograft.

P EMBODIMENT 18. The method of P EMBODIMENT 17, wherein inserting the second tissue implant comprises positioning the second tissue implant a distance away from a first tissue implant previously positioned within the segment.

P EMBODIMENT 19. The method of P EMBODIMENT 17 or P EMBODIMENT 18, further comprising piercing at least a portion of the trabecular meshwork spanning between the first tissue implant and the second tissue implant.

P EMBODIMENT 20. The method of P EMBODIMENT 15, wherein at least a distal end of the first tissue implant is located within Schlemm's canal and a proximal end of the first tissue implant is located within Schlemm's canal, within the trabecular meshwork, or within the anterior chamber.

P EMBODIMENT 21. The method of P EMBODIMENT 20, wherein the proximal end of the first tissue implant expands into the trabecular meshwork and/or into the anterior chamber upon rehydration and expansion following deployment.

P EMBODIMENT 22. The method of any one of P EMBODIMENTS 15-21, wherein dilating at least the segment of Schlemm's Canal comprises injecting a fluid into the segment.

P EMBODIMENT 23. The method of P EMBODIMENT 22, wherein the fluid comprises an ophthalmic viscoelastic solution.

P EMBODIMENT 24. A treatment system comprising one or more of a Schlemm's Canal dilation system and a tissue implant delivery system.

P EMBODIMENT 25. The treatment system of P EMBODIMENT 24, wherein the Schlemm's Canal dilation system comprises a dilation tool comprising a proximal hand piece and an outer shaft coupled to and extending distal from the proximal hand piece.

P EMBODIMENT 26. The treatment system of P EMBODIMENT 25, wherein the outer shaft is a tubular element having an inner lumen.

P EMBODIMENT 27. The treatment system of P EMBODIMENT 25 or P EMBODIMENT 26, wherein the outer shaft incorporates a straight proximal length extending along a longitudinal axis of the proximal hand piece and a curved distal length that curves away from the longitudinal axis.

P EMBODIMENT 28. The treatment system of P EMBODIMENT 27, wherein the curved distal length of the outer shaft is about 15-20 mm long and wherein the straight proximal length of the outer shaft is about 50-55 mm long.

P EMBODIMENT 29. The treatment system of P EMBODIMENT 27 or P EMBODIMENT 28, wherein the curved distal length incorporates a dual curve geometry.

P EMBODIMENT 30. The treatment system of any one of P EMBODIMENTS 25-29, wherein the outer shaft comprises a distal tip forming a distal opening that has a heel, a distal point, and a length between the heel and the distal point.

P EMBODIMENT 31. The treatment system of P EMBODIMENT 30, wherein the distal tip is shaped to form a spatula.

P EMBODIMENT 32. The treatment system of P EMBODIMENT 30 or P EMBODIMENT 31, wherein the distal tip comprises a first region forming a first curve and a second region forming a second curve and an inflection between a convex shape of the first curve and a concave shape of the second curve.

P EMBODIMENT 33. The treatment system of any one of P EMBODIMENTS 24-32, wherein the Schlemm's Canal dilation system further comprises a fluid delivery device.

P EMBODIMENT 34. The treatment system of any one of P EMBODIMENTS 24-33, wherein the tissue implant delivery system comprises a delivery cannula; an inner member positioned internal to the delivery cannula; and a tissue implant held within the delivery cannula distal to the inner member.

P EMBODIMENT 35. The treatment system of P EMBODIMENT 34, wherein the tissue implant comprises minimally-modified scleral allograft tissue.

P EMBODIMENT 36. The treatment system of P EMBODIMENT 34 or P EMBODIMENT 35, wherein a distal end region of the delivery cannula is flexible.

P EMBODIMENT 37. The treatment system of P EMBODIMENT 36, wherein the distal end region of the delivery cannula takes on a curved shape of the inner member.

P EMBODIMENT 38. The treatment system of P EMBODIMENT 37, wherein the distal end region of the delivery cannula comprises a beveled distal opening.

P EMBODIMENT 39. The treatment system of P EMBODIMENT 38, wherein a heel of the beveled distal opening is located on a radially-inward side of the curved shape.

P EMBODIMENT 40. The treatment system of P EMBODIMENT 38, wherein a heel of the beveled distal opening is located on a radially-outward side of the curved shape creating a backward bevel direction.

P EMBODIMENT 41. The treatment system of P EMBODIMENT 40, wherein the backward bevel direction aids in preserving the trabecular meshwork upon insertion of the distal end region into Schlemm's Canal.

P EMBODIMENT 42. The treatment system of any one of P EMBODIMENTS 34-41, wherein the tissue implant delivery system further comprises at least one actuator.

P EMBODIMENT 43. The treatment system of P EMBODIMENT 42, wherein the at least one actuator comprises a two-part actuator for deployment of the tissue implant from the delivery cannula.

P EMBODIMENT 44. The treatment system of any one of P EMBODIMENTS 34-43, wherein the at least one actuator is configured to be pushed distally to retract the delivery cannula.

P EMBODIMENT 45. The treatment system of any one of P EMBODIMENTS 34-44, wherein the inner member is fixed and the delivery cannula movable relative to the inner member.

1-13. (canceled)

14. A method of increasing aqueous outflow through Schlemm's Canal, the method comprising dilating at least a segment of Schlemm's Canal to stretch at least a portion of a trabecular meshwork; and inserting at least a first tissue implant within the segment of Schlemm's Canal, the first tissue implant being minimally-modified scleral allograft.

15. (canceled)

16. The method of claim 14, wherein the first tissue implant expands upon deployment within the segment by more than 5% in any direction upon hydration to increase aqueous outflow from an anterior chamber through Schlemm's Canal.

17. The method of claim 14, further comprising inserting a second tissue implant within the segment of Schlemm's Canal, the second tissue implant being minimally-modified scleral allograft.

18. The method of claim 17, wherein inserting the second tissue implant comprises positioning the second tissue implant a distance away from a first tissue implant previously positioned within the segment.

19. The method of claim 17, further comprising piercing at least a portion of the trabecular meshwork spanning between the first tissue implant and the second tissue implant.

20. The method of claim 14, wherein at least a distal end of the first tissue implant is located within Schlemm's canal and a proximal end of the first tissue implant is located within Schlemm's canal, within the trabecular meshwork, or within an anterior chamber.

21. The method of claim 20, wherein the proximal end of the first tissue implant expands into the trabecular meshwork and/or into an anterior chamber upon rehydration and expansion following deployment.

22. The method of claim 14, wherein dilating at least the segment of Schlemm's Canal comprises injecting a fluid into the segment.

23. The method of claim 22, wherein the fluid comprises an ophthalmic viscoelastic solution.

24. A treatment system comprising one or more of a Schlemm's Canal dilation system and a tissue implant delivery system.

25. The treatment system of claim 24, wherein the Schlemm's Canal dilation system comprises a dilation tool comprising a proximal hand piece and an outer shaft coupled to and extending distal from the proximal hand piece.

26. The treatment system of claim 25, wherein the outer shaft is a tubular element having an inner lumen.

27. The treatment system of claim 25, wherein the outer shaft incorporates a straight proximal length extending along a longitudinal axis of the proximal hand piece and a curved distal length that curves away from the longitudinal axis.

28. (canceled)

29. (canceled)

30. The treatment system of claim 25, wherein the outer shaft comprises a distal tip forming a distal opening that has a heel, a distal point, and a length between the heel and the distal point.

31. The treatment system of claim 30, wherein the distal tip has a shape of a spatula sharpened to penetrate a trabecular meshwork.

32. The treatment system of claim 30, wherein the distal tip comprises a first region forming a first curve and a second region forming a second curve and an inflection between a convex shape of the first curve and a concave shape of the second curve.

33. (canceled)

34. The treatment system of claim 24, wherein the tissue implant delivery system comprises a delivery cannula; an inner member positioned internal to the delivery cannula; and a tissue implant held within the delivery cannula distal to the inner member.

35. The treatment system of claim 34, wherein the tissue implant comprises minimally-modified scleral allograft tissue.

36-49. (canceled)

50. A device for dilating Schlemm's Canal, the device comprising:
a proximal hand piece comprising a slider;
an outer shaft coupled to the proximal hand piece comprising a lumen having a distal opening; and
an inner shaft positioned within the lumen of the outer shaft and movable relative to the outer shaft upon movement of the slider, wherein the inner shaft comprises a distal-most end,
wherein the distal-most end of the inner shaft is sized to dilate Schlemm's Canal as the distal-most end of the inner shaft is advanced through at least a portion of Schlemm's Canal forming a dilated canal segment, wherein a trabecular meshwork of the dilated canal segment remains intact.

51. The device of claim 50, wherein the inner shaft has a cross-sectional shape taken transverse to a length of the inner shaft, and wherein the cross-sectional shape is square or rectangular.

52. The device of claim 50, wherein the inner shaft comprises a first surface that is connected to a second surface by two lateral sides, wherein, in use, the first surface is an inward-facing surface and the second surface is an outward-facing surface.

53. The device of claim 52, wherein a region of the first surface located proximal of the distal-most end of the inner shaft comprises one or more visual markers, wherein, in use, the one or more visual markers indicate a degree of advancement of the inner shaft relative to Schlemm's Canal.

54. (canceled)

55. The device of claim 52, wherein the inner shaft further comprises a plurality of teeth located proximal of the distal-most end, wherein the plurality of teeth project from a region of the second surface.

56. The device of claim 55, wherein a thickness of the plurality of teeth along a lateral side from the first surface to the second surface is about 140-150 microns.

57. The device of claim 52, wherein a thickness of the distal-most end of the inner shaft along a lateral side from the first surface to the second is about 200-250 microns.

58. The device of claim 52, wherein the inner shaft further comprises a neck region located proximal of the distal-most end, wherein a thickness of the neck region along a lateral side from the first surface to the second surface is less than the thickness of the distal-most end.
